(19) United States
(12) Patent Application Publication
(10) Pub. No.: US 2020/0397523 A1
(43) Pub. Date: Dec. 24, 2020

(54) IMAGE-GUIDED MICROROBOTIC METHODS, SYSTEMS, AND DEVICES

(71) Applicant: California Institute of Technology, Pasadena, CA (US)

(72) Inventors: Wei Gao, Pasadena, CA (US); Lihong Wang, Arcadia, CA (US); Lei Li, Pasadena, CA (US); Zhiguang Wu, Harbin (CN)

(21) Appl. No.: 16/946,496

(22) Filed: Jun. 24, 2020

Related U.S. Application Data
(60) Provisional application No. 62/865,769, filed on Jun. 24, 2019.

(51) Int. Cl. A61B 34/00; A61B 90/00; A61K 9/50

(52) U.S. Cl. CPC A61B 34/72; A61B 90/36; A61K 9/5089; A61K 9/5078; A61B 2090/374; A61K 9/5057; A61K 9/5031; A61B 2090/3762; A61B 2090/378; A61K 9/5094

(57) ABSTRACT

Image-guided microrobotic systems, methods and methods that employ micromotor(s) having imaging agent(s) and cargo in a microcapsule, each micromotor having a partial coating over a reactive particle and/or asymmetrical geometry, when activated the microcapsule disintegrates releasing the micromotor(s) and active propulsion is generated when fluid contacts the reactive particle.

### CROSS-REFERENCE TO RELATED APPLICATION

This application claims priority to and benefit of U.S. Provisional Patent Application No. 62/865,769, titled “Photoacoustic Computed Tomography Guided Microrobotic System” and filed on Jun. 24, 2019, which is hereby incorporated by reference in its entirety and for all purposes.

### FEDERALLY SPONSORED RESEARCH OR DEVELOPMENT

This invention was made with government support under Grant No. CA186567 & NS090579 & NS099717 awarded by the National Institutes of Health. The government has certain rights in the invention.

### FIELD

Certain embodiments generally pertain to micromotors with imaging contrast agent(s) and cargo such as, for example, therapeutic drugs.

### BACKGROUND

Microrobots and nanorobots have drawn recent attention for their promise of enabling biomedical applications such as disease diagnosis, targeted drug delivery, and minimally-invasive and precise microsurgery. Some examples of microrobots/nanorobots can be found in Li, J., Esteban-Fernandez de Avila, B., Gao, W., Zhang, L., Wang, J., “Micro/nanorobots for biomedicine: Delivery, surgery, sensing, and detoxification,” Sci. Robot. 2, eaam 6431 (2017), Paxton, W. F., Kistler, K. C., Olmeda, C. C., Sen, A., St. Angelo, S. K., Cao, Y., Mallouk, T. E., Lammert, P. E., Crespi, V. H., “Catalytic nanomotors: Autonomous movement of striped nanorods,” J. Am. Chem. Soc. 126, 13424-13431 (2004), Hu, W., Lum, G. Z., Mastrangeli, M., Sitti, M., “Small-scale soft-bodied robot with multimodal locomotion,” Nature 554, 81-85 (2018), Fan, D., Yin, Z., Cheong, R., Zhu, F. Q., Cammarata, R. C., Chien, C. L., Levchenko, A., “Subcellular-resolution delivery of a cytokine through precisely manipulated nanowires,” Nat. Nanotechnol. 5, 545-551 (2010), Yan, X., Zhou, Q., Vincent, M. Deng, Y. Yu, J., Xu, J., Xu, T. Tang, T. Bian, L., Wang, J. Kostarelos, K. Zhang, L., “Multifunctional biohybrid magnetite microrobots for imaging-guided therapy,” Sci. Robot. 2, eaaq1155 (2017), and Hu, C., Pane, S., Nelson, B. J., “Soft micro- and nanorobotics,” Annu. Rev. Control. Robot. Auton. Syst. 1, 53-75 (2018), which are hereby incorporated by reference in their entireties.

### SUMMARY

A microrobotic device, comprising one or more micromotors and a microcapsule encapsulating the one or more micromotors. Each micromotor comprises a reactive particle and a partial coating disposed on the reactive particle. The partial coating comprises an imaging contrast layer, a cargo layer, and an encapsulation layer.

A method of fabricating a microrobotic device, the method comprising fabricating one or more micromotors, each micromotor fabricated by depositing a partial coating on a reactive particle, the partial coating comprising an imaging contrast material and cargo, the partial coating having one or more areas open to the reactive particle; and encapsulating the one or more micromotors in a microcapsule.

An image-guided microrobotic method, comprising using one or more images to determine that a microrobotic device is at or near a target region, wherein the microrobotic device comprises one or more micromotors encapsulated in a microcapsule, at least one of the micromotors comprising a partial coating disposed over a reactive particle, the partial coating comprising an imaging contrast material and cargo; and inducing disintegration of at least a portion of the microcapsule.

These and other features are described in more detail below with reference to the associated drawings.

### BRIEF DESCRIPTION OF THE DRAWINGS

FIG. 1 is a schematic drawing of components of an image-guided microrobotic system, according to an implementation.

FIG. 2 is a schematic drawing of components of the PACT system that can be employed in FIG. 1, according to an implementation.

FIG. 3A is a schematic drawing of a portion of the stomach of the mouse specimen in FIG. 1, according to an implementation.

FIG. 3B is a schematic drawing of a portion of the intestines of the mouse specimen in FIG. 1, according to an implementation.

FIG. 3C is a schematic drawing of a portion of the intestines of the mouse specimen in FIG. 1, according to an implementation.

FIG. 4 is a simplified block diagram of components of an image-guided microrobotic system, according to implementations.

FIG. 5 depicts a flowchart illustrating operations of a method of fabricating at least one image-guided microrobotic device, according to implementations.

FIG. 6 depicts a flowchart with an example of sub-operations of operation in FIG. 5, according to one aspect.

FIG. 7 is schematic drawing of operations of a fabrication flow for ingestible Mg-based micromotors, according to an implementation.

FIG. 8 is a scanning electron microscope image of an ingestible Mg-based spheroid micromotor fabricated using the operations described in FIG. 7, according to an implementation.

FIG. 9 depicts bright field and fluorescence microscopic images of the ingestible Mg-based micromotors fabricated using the operations described in FIG. 7, according to an implementation.

FIG. 10 is a schematic drawing depicting operations in an exemplary operation using the controlled emulsion technique for encapsulating micromotors in enteric gelatin microcapsules, according to an implementation.

FIG. 11 depicts a bar graph with rotational speeds of magnetic stirring for different diameters of image-guided microrobotic devices, according to an implementation.

FIG. 12 depicts microscopic images of three image-guided microrobotic devices with different diameters as formed by magnetic stirring at different rotational speeds, according to an implementation.

FIG. 13 depicts images of ingestible image-guided microrobotic devices with Mg-based micromotors, according to an implementation.

FIG. 14 depicts images of ingestible image-guided microrobotic devices with Mg-based micromotors, according to an implementation.

FIG. 15 depicts bright field and fluorescence microscopic images of the ingestible Mg-based micromotors fabricated using the operations described in FIG. 7, according to an implementation.

FIG. 16 are PACT images of bare Mg particles, whole blood, and the ingestible Mg-based image-guided microrobotic devices using laser wavelengths at 720, 750, and 870 nm respectively, according to an implementation.

FIG. 17 is a graph with plots of the measured PACT photoacoustic spectra of the ingestible Mg-based image-guided microrobotic devices, whole blood, and Mg particles, respectively.

FIG. 18A is a bar graph of normalized PA amplitude over time under NIR illumination used in PACT in vitro, according to an implementation.

FIG. 18B is a bar graph of normalized PA amplitude over time under NIR illumination used in PACT in vivo, according to an implementation.

FIG. 19 depict nine (9) gray-scaled versions of photoacoustic images using PACT for different increasing concentrations of micromotors, according to an implementation.

FIG. 20 depicts a graph with a plot of the PA amplitude vs. concentrations of micromotors and PA amplitude vs. fluence level of NIR illumination, according to an implementation.

FIG. 21 depicts a graph with a plot of the PA amplitude vs. depth of tissue for both image-guided microrobotic devices and blood and a plot of normalized fluorescence intensity vs. depth of tissue for both image-guided microrobotic devices and blood, according to an implementation.

FIG. 22A is a fluorescence image of the image-guided microrobotic devices in a silicone tube under chicken breast tissues with a thickness of 0 mm, according to an implementation.

FIG. 22B is a fluorescence image of the image-guided microrobotic devices in a silicone tube under chicken breast tissues with a thickness of 0.7 mm, according to an implementation.

FIG. 22C is a fluorescence image of the image-guided microrobotic devices in a silicone tube under chicken breast tissues with a thickness of 1.7 mm, according to an implementation.

FIG. 22D is a fluorescence image of the image-guided microrobotic devices in a silicone tube under chicken breast tissues with a thickness of 2.4 mm, according to an implementation.

FIG. 23 depicts a flowchart illustrating operations of an image-guided microrobotic method, according to implementations.

FIG. 24 is a schematic drawing of a silicon rubber tube modeled intestine sandwiched between two portions of chicken breast tissue, according to an implementation.

FIG. 25 depicts four time-lapsed photoacoustic images of the normalized photoacoustic amplitude taken by the PACT system at time=0 s, 3 s, 6 s, and 9 s, according to an implementation.

FIG. 26 is a schematic drawing illustrating activation on demand of propulsion of micromotors upon unwrapping from a microcapsule activated by high power CW NIR irradiation directed at a region with the image-guided microrobotic device, according to an implementation.

FIG. 27 includes two time-lapsed microscopic images showing the use of high power CW NIR irradiation to trigger the collapse of the microcapsule of an image-guided microrobotic device and the activation/propulsion of the micromotors, according to an implementation.

FIG. 28 includes two microscopic images of image-guided microrobotic devices in gastric acid and intestinal fluid to show their stability, according to one implementation.

FIG. 29 depicts two time-lapsed images taken at time=0 hour and 1 hour of an image-guided microrobotic device with an enteric coating and gelatin microcapsule in gastric acid to show stability, according to one implementation.

FIG. 30 depicts two time-lapsed images taken at time=0 hour and 8 hours of an image-guided microrobotic device with an enteric coating and gelatin microcapsule in intestinal fluid to show stability, according to one implementation.

FIG. 31A is a microscopic image showing the gas bubble propulsion of a micromotor 3110 in phosphate-buffered saline (PBS), according to an implementation.

FIG. 31B is a microscopic image showing the gas bubble propulsion of a micromotor in intestinal fluid, according to an implementation.

FIG. 31C is a bar graph showing the velocities of the micromotors in PBS and intestinal fluid, according to an implementation.

FIG. 32A is a microscopic image showing the behavior of an Mg microparticle in intestinal fluid, according to an implementation.

FIG. 32B is a microscopic image showing the behavior of an Mg microparticle in gastric acid, according to an implementation.

FIG. 32C is a bar graph of the velocities of the micromotors in intestinal fluid and gastric fluid, according to an implementation.

FIG. 33 includes gray-scaled versions of six (6) time-lapse PACT images of the image-guided microrobotic devices taken at time=0 hour, 1.5, hours, 3 hours, 4.5 hours, 6 hours, and 7.5 hours, according to an implementation.

FIG. 34A is a graph with a plot of the movement caused by the migration of the image-guided microrobotic devices in the intestine over time and a linear fit of the data, according to an implementation.

FIG. 34B is a graph with a plot of the image-guided microrobotic device movement over time by the respiration motion of the mouse and a linear fit of the data, according to an implementation.

FIG. 34C is a bar graph of a comparison of the speeds of the image-guided microrobotic devices migration and the respiration-induced movement.

FIG. 35 is a thresholded x-t image showing the segmented image-guided microrobotic devices at elapsed time, t, according to an example.

FIG. 36 is a graph of a plot of movement displacement caused by migration of the image-guided microrobotic devices in intestines, according to an example.

FIG. 37 is schematic drawing of an implementation of using an image-guided microrobotic method for targeted delivery of micromotors in intestines, according to an implementation.

FIG. 38 depicts two time-lapsed PACT images at time=0 and 4 seconds of the migration of an image-guided microrobotic device toward the model colon tumor, according to an implementation.

FIG. 39 depicts two images 1) first image with an image-guided microrobotic device before activation by WC NIR irradiation and 2) second image after activation by CW NIR irradiation, according to an implementation.

FIG. 40 depicts two overlaid microscope images one before activation by WC NIR irradiation and one after activation by CW NIR irradiation, according to an implementation.

FIG. 41A depicts three microscopic images showing the in vivo retention of the control microparticles and the micromotors in intestines, according to an implementation.

FIG. 41B is a bar graph of the density of particle micromotor retention in intestines of the micromotors, according to an implementation.

FIG. 42A is a microscopic image showing micromotors attached on the intestines before addition of 0.1 M gastric acid, according to an implementation.

FIG. 42B is a microscopic image showing micromotors attached on the intestines after addition of 0.1 M gastric acid, according to an implementation.

FIG. 43 is a microscopic image (lower left) and a schematic drawing (upper right) illustrating the change of pH of the surrounding environment upon the micromotors being released into PBS, according to an implementation.

FIG. 44 is a schematic drawing of the control silica particles and the ingestible micromotors in mucus after 1 hour, according to an implementation. The drawing shows the reaction Mg²⁺+OH⁻ at the micromotors.

FIG. 45 are the diffusion profiles of the control silica particles and the ingestible micromotors, according to an implementation.

FIG. 46A is a bar graph of encapsulation efficiency for control hydrogel and cross-linking hydrogel, according to an implementation.

FIG. 46B is a bar graph of encapsulation efficiency vs. DOX loading amount per micromotor, according to an implementation.

FIG. 47A is graph with a plot of DOX released percentage from image-guided microrobotic devices as a function of time, according to an implementation.

FIG. 47B is graph with a plot of DOX released percentage from micromotors as a function of time, according to an implementation.

FIG. 48 is a graph of the body weight changes in mice after oral administration of the image-guided microrobotic devices and the control (DI water) over time, according to an implementation.

FIG. 49 is a histology analysis for the duodenum, jejunum, and distal colon of the mice treated with the image-guided microrobotic devices or DI water as the control for 12 hours, according to an implementation.

### DETAILED DESCRIPTION

Different aspects are described below with reference to the accompanying drawings. The features illustrated in the drawings may not be to scale. In the following description, numerous specific details are set forth in order to provide a thorough understanding of the presented embodiments. The disclosed embodiments may be practiced without one or more of these specific details. In other instances, well-known operations have not been described in detail to avoid unnecessarily obscuring the disclosed embodiments. While the disclosed embodiments will be described in conjunction with the specific embodiments, it will be understood that it is not intended to limit the disclosed embodiments. For example, while certain techniques are described with reference to biomedical applications, it would be understood that these same techniques can be used to address environmental remediation, micro/nanofabrication, and detoxification. As another example, while certain techniques are described with reference to techniques that image and control micromotors, it would be understood that these same techniques can be used to image and control other microrobots such as magnetic propellers and also nanorobots.

### I. Introduction

Chemically-powered micromotors with autonomous propulsion and/or versatile functions in biofluids might prove to be particularly useful for in vivo applications. Some examples of conventional chemically-powered micro/nano motors can be found in Sanchez, S., Soler, L., and Katuri, J., “Chemically powered micro- and nanomotors,” Angew. Chem. Int. Ed. 54, 1414-1444 (2015), Tu, Y., Peng, F., Sui, X., Men, Y., White, P. B., van Hest, J. C. M., and Wilson, D. A., “Self-propelled supramolecular nanomotors with temperature-responsive speed regulation,” Nat. Chem. 9, 480 (2016), Esteban-Fernandez de Avila, B., Angsantikul, P., Li, J., Lopez-Ramirez, M. A., Ramirez-Herrera, D. E., Thamphiwatana, S., Chen, C., Delezuk, J., Samakapiruk, R., Ramez, V., Zhang, L., and Wang, J., “Micromotor-enabled active drug delivery for in vivo treatment of stomach infection,” Nat. Commun. 8, 272 (2017), Wang, J., Gao, W., “Nano/microscale motors: biomedical opportunities and challenges,” ACS Nano 6, 5745-5751 (2012), Gao, W., Dong, R., Thamphiwatana, S., Li, J., Gao, W., Zhang, L., and Wang, J., “Artificial micromotors in the mouse's stomach: A step toward in vivo use of synthetic motors,” ACS Nano 9, 117-123 (2015), which are hereby incorporated by reference in their entireties. Discussion of recent progress in micromotors can be found in Li, T., Chang, X., Wu, Z., Li, J., Shao, G., Deng, X., Qiu, J., Guo, B., Zhang, G., He, Q., Li, L., and Wang, J., “Autonomous collision-free navigation of microvehicles in complex and dynamically changing environments,” ACS Nano 11, 9268-9275 (2017), Sitti, M., “Miniature soft robots-road to the clinic,” Nat. Rev. Mater, 3, 74-75 (2018), Medina-Sanchez, and M. S., Schmidt, O. G., “Medical microbots need better imaging and control,” Nature 545, 406-408 (2017), Vilela, D., Cossío, U., Parmar, J., Martinez-Villacorta, A. M., Gomez-Vallejo, V., Llop, J. Sanchez, S., “Medical imaging for the tracking of micromotors,” ACS Nano 12, 1220-1227 (2018), which are hereby incorporated by reference in their entireties.

To date, optical imaging is widely used for biomedical applications owing to its high spatiotemporal resolution and molecular contrasts. However, applying conventional optical imaging to deep tissues is hampered by strong optical scattering, which inhibits high-resolution imaging beyond the optical diffusion limit (˜1-2 mm in depth) as discussed in Ntziachristos, V., “Going deeper than microscopy: the optical imaging frontier in biology,” Nat. Methods 7, 603-614 (2010), which is hereby incorporated by reference in its entirety. Fortunately, photoacoustic (PA) tomography (PAT), detecting photon-induced ultrasound, achieves high-resolution imaging at depths that far exceed the optical diffusion limit as discussed in Razansky, D., Distel, M., Vinegoni, C., Ma, R., Perrimon, N., Koster, R. W., Ntziachristos, V., “Multispectral opto-acoustic tomography of deep-seated fluorescent proteins in vivo,” Nat. Photonics 3, 412-417 (2009), which is hereby incorporated by reference in its entirety. In PAT, the energy of photons absorbed by chromophores inside the tissue is converted to acoustic waves, which are subsequently detected to yield high-resolution tomographic images with optical contrasts. Leveraging the negligible acoustic scattering in soft tissue, PAT has achieved superb spatial resolution at depths, with a depth-to-resolution ratio of ˜200, at high imaging rates, as discussed in Wang, L. V., Hu, S., “Photoacoustic tomography: in vivo imaging from organelles to organs,” Science 335, 1458-1462 (2012), which is hereby incorporated by reference in its entirety.

Also, photoacoustic computed tomography (PACT) has been able to attain high spatiotemporal resolution (125-μm in-plane resolution and 50-μs frame⁻¹ data acquisition), deep penetration (48-mm tissue penetration in vivo), and anatomical and molecular contrasts as discussed in Li, L., Zhu, L., Ma, C., Lin, L., Yao, J., Wang, L., Maslov, K., Zhang, R., Chen, W., Shi, J., “Single-impulse panoramic photoacoustic computed tomography of small-animal whole-body dynamics at high spatiotemporal resolution,” Nat. Biomed. Eng. 1, 0071 (2017), Li, L., Shemetov, A. A., Baloban, M., Hu, P., Zhu, L., Shcherbakova, D. M., Zhang, R., Shi, J., Yao, J., Wang, L. V., Verkhusha, V. V., “Small near-infrared photochromic protein for photoacoustic multi-contrast imaging and detection of protein interactions in vivo,” Nat. Commun. 9, 2734 (2018), Yao, J., Kaberniuk, A. A., Li, L., Shcherbakova, D. M., Zhang, R., Wang, L., Li, G., Verkhusha, V. V., Wang, L. V., “Multiscale photoacoustic tomography using reversibly switchable bacterial phytochrome as a near-infrared photochromic probe,” Nat. Methods 13, 67 (2015), which are hereby incorporated by reference in their entireties.

Previously, small-animal whole-body imaging typically relied on non-optical approaches such as, e.g., X-ray computed tomography (X-ray CT), magnetic resonance imaging (MRI), positron emission tomography (PET) or single-photon emission computed tomography (SPECT), and ultrasound imaging (USI) as discussed in Ntziachristos, V., “Going deeper than microscopy: the optical imaging frontier in biology,” Nat. Methods 7, 603-614 (2010), which is hereby incorporated by reference in its entirety. Although these non-optical techniques provided deep penetration, they suffer from significant limitations. For example, microscopic MRI requires a long data acquisition time, ranging from seconds to minutes, too slow for imaging dynamics as discussed in Wu, D., Zhang, J., “In vivo mapping of macroscopic neuronal projections in the mouse hippocampus using high-resolution diffusion MRI,” NeuroImage 125, 84-93 (2016) and Alomair, O. I., Brereton, I. M., Smith, M. T., Galloway, G. J., Kurniawan, N. D., “In vivo high angular resolution diffusion-weighted imaging of mouse brain at 16.4 Tesla,” PloS One 10, e0130133 (2015), which are hereby incorporated by reference in their entireties. More importantly, MRI, requiring a strong magnetic field, is incompatible with magnetically driven or guided micromotors as discussed in Yan, X., Zhou, Q., Vincent, M. Deng, Y. Yu, J., Xu, J., Xu, T. Tang, T. Bian, L., Wang, J. Kostarelos, K. Zhang, L., “Multifunctional biohybrid magnetite microrobots for imaging-guided therapy,” Sci. Robot. 2, eaaq1155 (2017), which is hereby incorporated by reference in its entirety. X-ray CT has poor contrast of the micromotors made of biocompatible/biodegradable metals as discussed in Vilela, D., Cossío, U., Parmar, J., Martinez-Villacorta, A. M., Gomez-Vallejo, V., Llop, J. Sanchez, S., “Medical imaging for the tracking of micromotors,” ACS Nano 12, 1220-1227 (2018) and Schambach, S. J., Bag, S., Schilling, L., Groden, C., Brockmann, M. A., “Application of micro-CT in small animal imaging,” Methods 50, 2-13 (2010), which are hereby incorporated by reference in their entireties. PET/SPECT alone suffers from poor spatial resolution. In addition, X-ray CT and PET/SPECT employ ionizing radiation, which inhibits longitudinal monitoring as discussed in Brenner, D. J., Hall, E. J., “Computed tomography—an increasing source of radiation exposure,” N. Engl. J. Med 357, 2277-2284 (2007), which is hereby incorporated by reference in its entirety. USI does not image extravascular molecular contrasts as discussed in Brenner, D. J., Hall, E. J., “Computed tomography—an increasing source of radiation exposure,” N. Engl. J. Med 357, 2277-2284 (2007), which is hereby incorporated by reference in its entirety. In addition, the microcapsules (MCs) of certain aspects described herein are mainly by mass composed of gelatin, which has almost the same acoustic impedance as soft tissue as discussed in Lai, P., Xu, X., Wang, L. V., “Dependence of optical scattering from Intralipid in gelatin-gel based tissue-mimicking phantoms on mixing temperature and time,” J Biomed. Opt. 19, 035002 (2014), which is hereby incorporated by reference in its entirety. Thus, USI cannot image microcapsules with sufficient contrast in vivo. Optical imaging uses non-carcinogenic electromagnetic waves to provide extraordinary molecular contrasts with either endogenous or exogenous agents at high spatiotemporal resolution. Unfortunately, the strong optical scattering of tissue hampers the application of conventional optical imaging technologies to small-animal whole body imaging at high spatial resolution as discussed in V. Ntziachristos, Going deeper than microscopy: the optical imaging frontier in biology. Nat. Methods 7, 603-614 (2010), which is hereby incorporated by reference in its entirety. On the other hand, photoacoustic tomography (PAT) can break the optical diffusion limit (as discussed in D. Razansky, M. Distel, C. Vinegoni, R. Ma, N. Perrimon, R. W. Koster, V. Ntziachristos, “Multispectral opto-acoustic tomography of deep-seated fluorescent proteins in vivo,” Nat. Photonics 3, 412-417 (2009), which is hereby incorporated by reference in its entirety) on penetration and achieves high-resolution imaging in deep tissues with optical contrasts.

Similarly, photoacoustic computed tomography (PACT) can attain high spatiotemporal resolution, deep penetration, and anatomical and molecular contrasts. Typically when implementing PACT to image tissue, a laser pulse is used to broadly illuminate the whole tissue sample to be imaged. As photons propagate inside the tissue, some are absorbed by molecules, and their energy is partially or completely converted into heat, creating a temperature rise through nonradiative relaxation. The local temperature rise induces a pressure rise through thermoelastic expansion. The pressure rise propagates, at a speed of roughly 1500 m s⁻¹, inside the tissue as a photoacoustic wave, and is detected outside the tissue by an ultrasonic transducer or transducer array. The detected photoacoustic signals are processed by a computing device to form an image, which maps the original optical energy deposition in the biological tissue. Because ultrasound scattering in soft tissue is about three orders of magnitude weaker than light scattering on a per unit path length basis in the ultrasonic frequency of interest, PACT may achieve superb spatial resolution at depths by detecting ultrasound.

Drug delivery through the gastrointestinal (GI) tract serves as a convenient and versatile therapeutic tool, owing to its cost-effectiveness, high patient compliance, lenient constraint for sterility, and ease of production as discussed in Bellinger, A., et al. “Oral, ultra-long-lasting drug delivery: application toward malaria elimination goals,” Sci. Transl. Med. 8, 365ra157 (2016) and Koziolek, M., et al., “Navigating the human gastrointestinal tract for oral drug delivery: Uncharted waters and new frontiers,” Adv. Drug Delivery Rev. 101, 75-88 (2016), which are hereby incorporated by reference in their entireties. Drug absorption of conventional micro/nanoparticle-based drug delivery systems is inefficient due to the limited intestinal retention time as discussed in Soppimath, K. S., Kulkarni, A. R., Rudzinski, W. E, Aminabhavi, T. M., “Microspheres as floating drug-delivery systems to increase gastric retention of drugs,” Drug Metab. Rev. 33, 149-160 (2001), which is hereby incorporated by reference in its entirety. Passive diffusion-based targeting strategies have been explored to improve delivery efficiency, but they suffer from low precision, size restraint and specific surface chemistry as discussed in Rosenblum, D., Joshi, N., Tao, W., Karp, J. M., Peer, D., “Progress and challenges towards targeted delivery of cancer therapeutics,” Nat. Commun. 9, 1410 (2018), which is hereby incorporated by reference in its entirety. Also, conventional microrobotic systems do not have precise control of microrobots in vivo as discussed in Yang, G.-Z., et al. “The grand challenges of science robotics,” Sci. Robot. 3, eaar7650 (2018) and Medina-Sanchez, M. S., Schmidt, O. G., “Medical microbots need better imaging and control,” Nature 545, 406-408 (2017), which are hereby incorporated by reference in their entireties. Additionally, biodegradability and biocompatibility are required, and an ideal microrobotic system is expected to be cleared safely by the body after completion of the tasks as discussed in Yan, X., Zhou, Q., Vincent, M. Deng, Y. Yu, J., Xu, J., Xu, T. Tang, T. Bian, L., Wang, J. Kostarelos, K. Zhang, L., “Multifunctional biohybrid magnetite microrobots for imaging-guided therapy,” Sci. Robot. 2, eaaq1155 (2017), Abdelmohsen, L. K. E. A., Peng, F., Tu, Y., Wilson, D. A., “Micro- and nano-motors for biomedical applications,” J. Mater. Chem. B 2, 2395-2408 (2014), and Wang, H., Pumera, M., “Fabrication of micro/nanoscale motors,” Chem. Rev. 115, 8704-8735 (2015), which are hereby incorporated by reference in their entireties.

### II. Image-Guided Microrobotic Techniques

Certain aspects pertain to image-guided microrobotic techniques (e.g., systems, methods, and devices) that employ imaging to navigate microrobots, such as micromotors, in deep tissue at high spatiotemporal resolution and high contrast, and with precise on-demand control of the microrobots, particularly in in vivo applications. In one aspect, for example, photoacoustic computed tomography (PACT) is employed to monitor and navigate micromotors in vivo, e.g., through the intestines. Some examples of PACT systems and methods that can be employed are described in U.S. patent application Ser. No. 16/798,204, titled “PHOTOACOUSTIC COMPUTED TOMOGRAPHY (PACT) SYSTEMS AND METHODS,” filed on Feb. 21, 2020 and Lin, L., Hu, P., Shi, J. et al., “Single-breath-hold photoacoustic computed tomography of the breast,” Nat. Commun. 9, 2352 (2018), which are hereby incorporated by reference in their entireties. Owing to the high spatiotemporal resolution, non-invasiveness, molecular contrast, and deep penetration aspects of PACT, it can be an attractive tool for imaging and navigating micromotors in deep tissue in vivo. Other examples of imaging techniques that can be used to image and guide micromotors are ultrasound, magnetic resonance imaging (MRI), X-ray computed tomography (CT), positron emission tomography (PET), diffuse optical tomography (DOT), photoacoustic microscopy (PAM), optical coherent tomography (OCT).

Certain implementations pertain to image-guided microrobotic devices. An image-guided microrobotic device includes, at least in part, one or more micromotors and a microcapsule (also sometimes referred to herein as “micromotor capsule”) enveloping the one or more micromotors. Once released from the microcapsule, the micromotor(s) can exhibit propulsion in various fluids such as, e.g., biofluids. In one aspect, the image-guided microrobotic device is spheroid or spherical and has a diameter in the range of about 20 μm to about 1000 μm.

In certain implementations, a micromotor includes a partial coating deposited on or otherwise disposed on a reactive particle or other form of reactive material(s) (also sometimes referred to herein as a “reactive core”). Although the reactive particle is generally spheroid or spherical, other shapes can be used such as oblong or cylinder. Some examples of reactive materials that can be used include magnesium, zinc, sodium carbonate. Some examples of the rough diameters of an approximately spherical reactive particle include 20±5 μm, 60±10 μm, 3±0.5 μm, and 100±20 μm. In one aspect, the rough diameter of an approximately spherical reactive particles is in a range of 20 μm to 60 μm. In one aspect, the reactive core is a magnesium microparticle. An example of a suitable commercially-available magnesium microparticle is the magnesium microparticle with a diameter of 20±5 μm sold by TangShan WeiHao Magnesium Powder. The partial coating of a micromotor includes one or more material layers that have imaging contrast agent(s) and a cargo carrier material with cargo such as, e.g., one or more therapeutic drugs, imaging contrast agents, photodynamic particles, and/or magnetic particles. In one aspect, at least one material layer of the partial coating that has both an imaging contrast agent(s) and cargo. In contrast with conventional microrobots, micromotors of certain implementations described herein employ a biocompatible propulsion mechanism, e.g., from a reaction between magnesium and water, to implement efficient and biocompatible self-propulsion in various biofluids such as gastric and intestinal fluids.

Although the image-guided microrobotic techniques described herein are mainly described with reference to micromotors, it would be understood that these techniques apply to other microrobots such as acoustically powered microrobots. Some examples of materials that can be employed as an imaging contrast agent include, for example, micro/nanoparticles, organic dyes, reporter gene proteins, microbubbles, fluorescent molecules, quantum dots, and metals. Some examples of materials that can be employed as a controllable cargo carrier include, for example, mesoporous silica, metal of framework, microcapsule, and polymersome.

In certain implementations, a micromotor includes a reactive particle at least partially coated by: 1) an imaging contrast layer, 2) a cargo layer, and/or 3) an encapsulation layer. The imaging contrast layer includes one or more imaging contrast agents. Some examples of imaging contrast agents include metals such as gold (Au), an organic dye, reporter gene protein, micro/nanoparticles, and/or microbubbles. In one example, the imaging contrast layer is a layer of gold (Au) having an approximate thickness of 50 nm. In one aspect, the thickness of the imaging contrast layer is in a range of 1 μm and 20 μm. In another aspect, the thickness of the imaging contrast layer is in a range of 10 μm and 20 μm. The imaging contrast layer may be used to increase optical absorption of the micromotor for imaging purposes (e.g., for photoacoustic imaging) and/or increase the reaction rate of the reactive core for efficient propulsion. For example, employing an Au layer as the imaging contrast layer over a magnesium particle can both increase the optical absorption of the micromotor for photoacoustic imaging and increase the reaction rate of the magnesium particle for efficient propulsion simultaneously. The material composition of the cargo layer may be designed to increase the loading capacity of the functional components (also referred to herein as “cargo”) such as, e.g., therapeutic drugs and imaging contrast agents. The cargo layer includes a cargo carrier material and cargo. The material composition of the encapsulation layer may be designed to maintain the geometry of the micromotor during propulsion. For example, the encapsulation layer may include parylene.

The partial coating of the micromotor includes one or more areas open to the reactive particle, which allow ingress of fluid that may react with the reactive particle. In certain implementations, the one or more open areas are located on one side (e.g., a portion of an outer surface of the micromotor that is facing substantially one direction) of the microrobot. For example, the micromotor may have a partial coating with one or more open areas on one side that are open to a reactive magnesium particle at the center of the micromotor. In one aspect, the size of an open area in a partial coating is in a range of 5 μm² to 500 μm². In one aspect, the size of the open area in a partial coating is less than 100 μm². According to implementations where micromotors are released into a body e.g. into the intestines, when the micromotors are released from the microcapsule, biofluids can pass through an open area to the reactive core (e.g., magnesium particle) and gas bubbles may be generated and/or cargo released. As gas bubbles exit the open area on one side, a propulsion force is created in a direction opposite the direction that the side faces.

In another aspect, micromotors may have a geometry that can generate directional bubbles to provide propulsion force in one direction without implementing a partial coating. For example, a micromotor may have a cylindrical structure with an asymmetrical opening on end of the cylindrical structure. A catalyst encapsulated in inside the structure can react with fluid to generate the bubbles that exit the open end providing the propulsive force.

In certain aspects, an image-guided microrobotic device includes one or more micromotors and a microcapsule that envelopes the one or more micromotors. The microcapsule may be formed from material(s) that are stable and protect the one or micromotors from the environment outside the microcapsule while the image-guided microrobotic device is travelling to the region being targeted for deployment. For example, the microcapsule may include a protective material that is capable of being stable in gastric fluids in the stomach while the image-guided microrobotic device travels to the intestines. Some examples of materials that may be used to form the microcapsule include, for example, gelatin material, enteric polymer, and/or parylene.

In one implementation, the partial coating of a micromotor includes a magnetically-charged material such as, e.g., iron oxide, nickel, and/or iron. In this case, the direction of the micromotor can be magnetically controlled by using external alternative magnetic field. An example of using magnetic control to control a microrobot can be found in Servant, A. et al. “Controlled in vivo swimming of a swarm of bacteria-like microrobotic flagella,” Advanced Materials 27, 2981-2988(2015), which is hereby incorporated by reference in its entirety.

In one exemplary method, one or more image-guided microrobotic devices, each with one or more micromotors encapsulated in a microcapsule, are ingested, injected, or otherwise introduced into the specimen. The material composition the microcapsule is stable in the environment into which it is introduced such as, e.g., in the gastric fluids of the stomach if ingested. The migration of the one or more image-guided microrobotic devices toward the targeted region (e.g., through the intestines to a tumor) can been visualized in real time in vivo by PACT or another imaging method. Once it is determined, using the images acquired, that the one or more microcapsules have arrived at or near the targeted region, a release trigger, such as, e.g., near-infrared light irradiation, high-intensity focused ultrasound, and/or magnetic field, induces disintegration or collapse of the one or more microcapsules to discharge the cargo-loaded micromotors. Once released, the reactive cores of the one or more micromotors are exposed to the biofluids or other fluids that may produce gases that generate propulsion when exiting through one or more open areas in the micromotors. The propulsion of the microrobots may effectively prolong the retention of the one or more micromotors in the specimen, and more particularly, in the targeted area. The image-guided microrobotic method may enable deep imaging and precise control of the one or more micromotors in vivo for biomedical applications such as, e.g., drug delivery and microsurgery.

Certain aspects pertain to imaging-guided microrobotic devices, systems, and methods that allow deep tissue navigation and placement of micromotors with enhanced retention in vivo. The imaging-guided microrobotic devices may be ingestible or injectable in some cases. For example, some image-guided microrobotic techniques may be operable to directly visualize the dynamics of one or more micromotors with high spatiotemporal resolution in vivo at the whole-body scale to provide real-time visualization and guidance of the one or more micromotors. In addition to high spatiotemporal resolution, these image-guided microrobotic techniques may also provide deep penetration and molecular contrast. According to one aspect, an image-guided microrobotic technique implements photoacoustic computed tomography (PACT) to visualize and/or guide a plurality of micromotors in one or more microcapsules to a targeted region. According to one aspect, the imaging-guided microrobotic devices, systems, and methods enable controlled propulsion of micromotors and prolonged cargo retention in vivo.

In one implementation, an imaging-guided microrobotic system includes one or more imaging-guided microrobotic devices that are ingestible for imaging-assisted control in intestines. Each imaging-guided microrobotic device includes a microcapsule that encapsulates one or more micromotors. The encapsulated micromotors survive the erosion of the stomach fluid and to allow for release and propulsion in the intestines. In some cases, PACT is employed to non-invasively monitor the migration of one or more imaging-guided microrobotic devices and/or the released micromotors, and visualizes their arrival at targeted areas in vivo. Continuous-wave near infrared radiation (CW NIR) is directed toward the targeted region to induce disintegration of the microcapsules at or near the target region, which triggers the propulsion of the micromotors. For example, if the microcapsule is gelatin-based and the imaging contrast layer is an Au layer, the Au layer can convert the NIR light to heat resulting in gel-sol phase transition of the gelatin-based microcapsule. The mechanical propulsion provides a driving force for the micromotors to be able to bind to the intestine walls, which may result in prolonged retention of the micromotors and/or their cargo in the tissues of the targeted region.

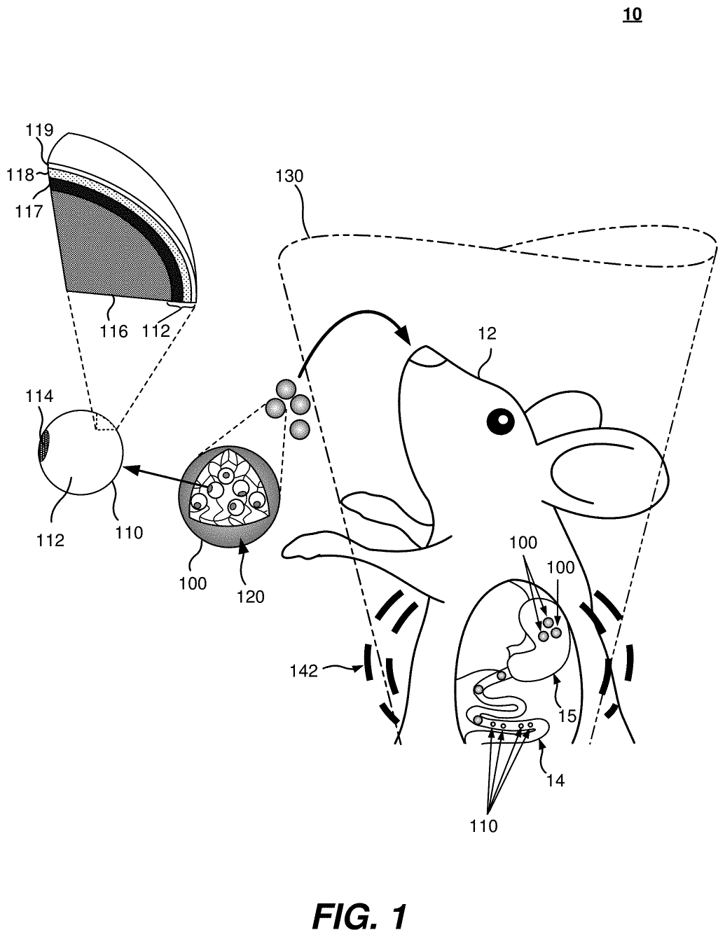

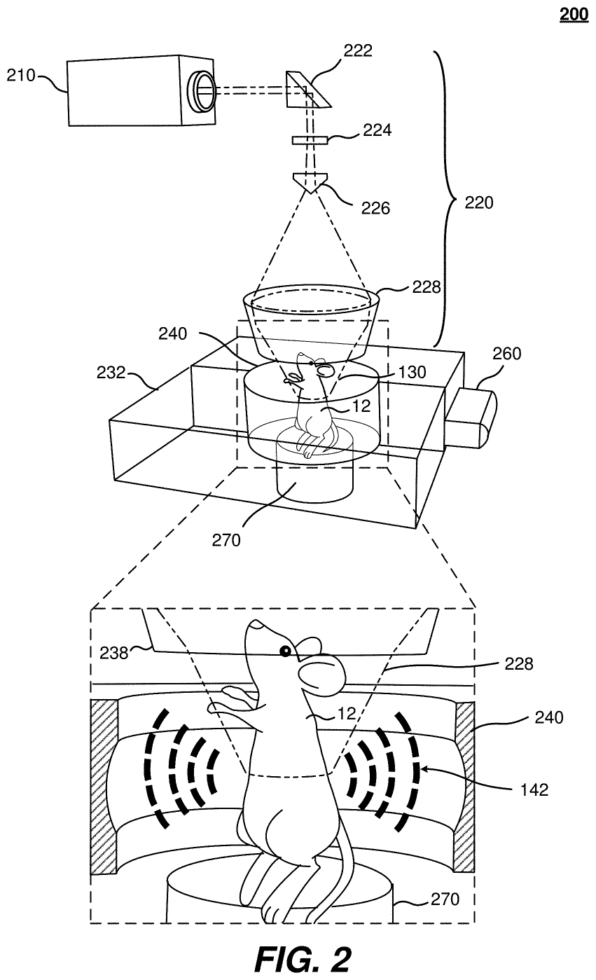

FIG. 1 is a schematic drawing of components of an image-guided microrobotic system 10, according to an implementation. The image-guided microrobotic system 10 includes a plurality of image-guided microrobotic devices 100. Each image-guided microrobotic device 100 includes one or more micromotors 110 and a microcapsule 120 encapsulating the one or more micromotors 110. In this implementation, the plurality of image-guided microrobotic devices 100 have been administered to (e.g., ingested by) a mouse specimen 12 and a PACT system 200 (shown in part in FIG. 2) is employed to image the image-guided microrobotic devices 100 in order to monitor the location of the image-guided microrobotic devices 100 as they pass through at least a portion of the digestive tract including the stomach 14 and intestines 15 of the mouse specimen 12. Owing to the high spatiotemporal resolution, non-invasiveness, molecular contrast, and deep penetration, PACT may be an attractive tool for imaging the image-guided microrobotic devices and/or micromotors in vivo as discussed in Wang, L. V., and Hu, S., “Photoacoustic tomography: in vivo imaging from organelles to organs,” Science 335, 1458-1462 (2012), Li, L. et al., “Single-impulse panoramic photoacoustic computed tomography of small-animal whole-body dynamics at high spatiotemporal resolution,” Nat. Biomed. Eng. 1, 0071 (2017), and Li, L., et al., “Small near-infrared photochromic protein for photoacoustic multi-contrast imaging and detection of protein interactions in vivo,” Nat. Commun. 9, 2734 (2018), which are hereby incorporated by reference in their entireties. In other implementations, other imaging techniques may be used. In other implementations, other imaging techniques can be employed.

Returning to FIG. 1, an image-guided microrobotic device 100 is shown with a portion of the microcapsule (MC) 120 cut away to show the plurality of micromotors 110 encapsulated therein. In this implementation, the microcapsule 120 is an enteric protective capsule that is gelatin-based and can protect the micromotors 110 from the gastric acid in the stomach 14 and allow direct visualization using PACT. The capsule encapsulating micromotors provides strong contrast for direct visualization PACT. Also shown is an expanded view (denoted by an arrow) of one of the micromotors 110 in the microcapsule 120. In the expanded view, the micromotor 110 is shown to include a partial coating 112 with an open area 114 on one side of the micromotor 110. A portion of the micromotor 110 is shown in another expanded view. In this second expanded view, the micromotor 110 is shown to include a reactive core 116 that is a magnesium particle and a partial coating 112 disposed on the reactive core 116. The reactive core 116 is exposed at the open area 114 of the micromotor 110. The partial coating 112 has a plurality of layers including: an imaging contrast layer 117, a cargo layer 118, and an encapsulating layer 119. In one aspect, the imaging contrast layer 117 includes gold (Au). The cargo layer 118 includes cargo such as, e.g., one or more drugs and/or one or more imaging contrast agents. In one aspect, the encapsulating layer 119 is a parlyene layer.

In FIG. 1, three image-guided microrobotic devices 100 are shown to have been administered to (e.g., ingested by) the mouse specimen 12. Each image-guided microrobotic device 100 includes one or more micromotors 110 encapsulated in enteric protective microcapsules 120 to prevent reactions in gastric acid in the stomach 14 and allow direct visualization by a PACT system 200 (shown in FIG. 2). In this implementation, the PACT system 200 is configured to monitor the migration of three image-guided microrobotic devices 100 in real time to determine approximately when the one or more image-guided microrobotic devices 100 are at or near a targeted region such as, e.g., diseased tissue. A light source of the PACT system 200 emits light pulses and an optical system 130 generates illumination to illuminate the mouse specimen 12. The photons propagate inside the tissue, some are absorbed by molecules, and their energy is partially or completely converted into heat, creating a temperature rise through nonradiative relaxation. The local temperature rise induces a pressure rise through thermoelastic expansion that propagates as a photoacoustic wave 142. A continuous wave (CW) near-infrared (NIR) light irradiation may be used to induce phase transition of the gelatin-based microcapsules 120, which releases and triggers propulsion of the micromotors 110. Other triggering mechanisms can be used in other implementations such as, e.g., implantable devices, endoscopic instrument, and therapeutic pill. The mechanical propulsion provides a driving force for the micromotors 110 to enter and/or bind to the intestine walls, which may result in prolonged retention of the micromotors 110 and/or their cargo in the tissues of the targeted region.

FIG. 2 is a schematic drawing of components of the PACT system 200 that can be employed in FIG. 1, according to an implementation. The PACT system 200 includes a light source 210 in the form of a pulsed 1064 nm laser source and an optical system 220 in optical communication with the light source 210 to receive laser pulses during operation. The optical system 220 includes a prism 222 in optical communication with the light source 210 to receive light pulses, and an axicon lens 224, an engineered diffuser 226 configured to convert the light pulses into a donut beam, and an optical condenser 228 configured to converge the donut beam. In addition, the PACT system 200 includes a tank 232 with an acoustic medium such as water within which the mouse specimen 12 is located during image acquisition. The PACT system 200 also includes an ultrasonic transducer array 240 (e.g., a 512-element full-ring ultrasonic transducer array) and a scanner 270 coupled to the ultrasonic transducer array 240 configured to move the ultrasonic transducer array 240 to one or more elevational positions and/or scan the ultrasonic transducer array 240 between two elevational positions along a z-axis (not shown). In one aspect, the ultrasonic transducer array 240 is a 512-element full-ring ultrasonic transducer array with a 220 mm ring diameter, 2.25 MHz central frequency, and more than 95% one-way bandwidth and/or each transducer element has a flat-rectangular aperture (e.g., 5 mm element elevation size; 1.35 mm pitch; and 0.7 mm inter-element spacing). The PACT system 200 also includes one or more data acquisition subsystems 260. Each data acquisition subsystem 260 includes a 128-channel preamplifier and a 128-channel data acquisition subsystem (e.g., Sonix DAQ made by Ultrasonix Medical ULC with a 40 MHz sampling rate and 12-bit dynamic range with programmable amplification up to 51 dB) in electrically communication with each other, e.g., in one-to-one mapping associations. The 128-channel preamplifier(s) are in electrically communication with the ultrasonic transducer array 240 via signal cable bundles. The PACT system 200 also includes a translational stage 270 configured to move along a z-axis to one or more elevational positions and hold for a time period or to scan between two elevational positions.

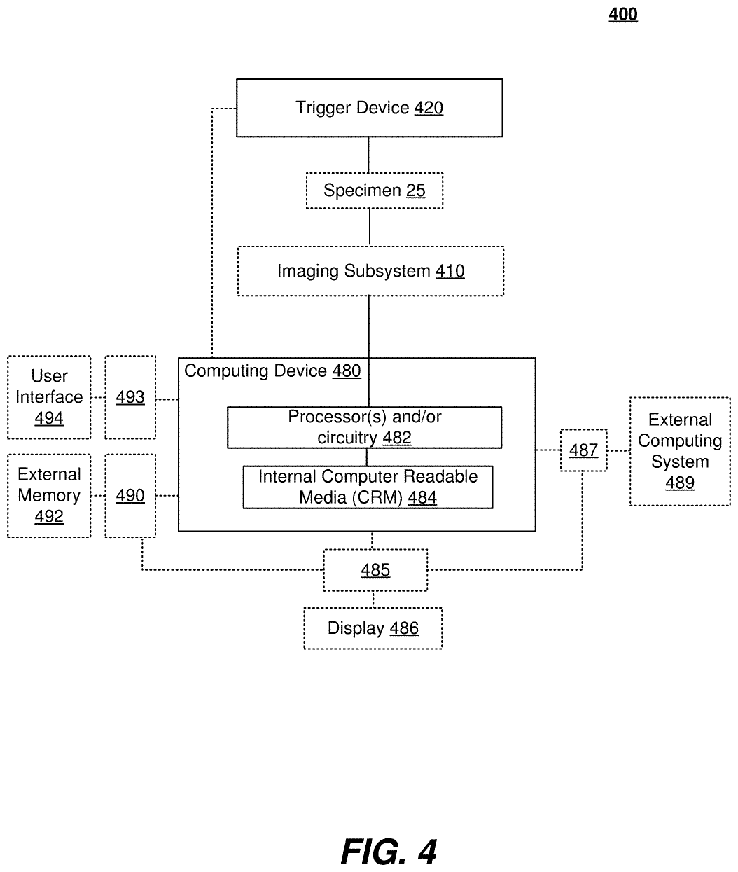

During image acquisitions operations of the PACT system 200, the light source 210 is triggered to emit light pulses and illumination is generated to illuminate the mouse specimen 12. As photons propagate inside the tissue, some are absorbed by molecules, and their energy is partially or completely converted into heat, creating a temperature rise through nonradiative relaxation. The local temperature rise induces a pressure rise through thermoelastic expansion. The pressure rise propagates inside the tissue as a photoacoustic wave 242, and is detected outside the tissue by the ultrasonic transducer array 240. The translational stage 270 may be moved to one or more elevational positions and held for a time period or scanned between two elevational positions during image acquisition to capture images at different planes. The detected photoacoustic signals are processed by the computing device (e.g., the computing device 480 shown in FIG. 4) to construct one or more photoacoustic images, which map the original optical energy deposition in the biological tissue. During image acquisition, the mouse specimen 12 was kept in the tank 232 surrounded by the elevationally-focused ultrasound transducer array 240.

In certain aspects, the imaging system such as a PACT system takes time-lapsed images as the image-guided microrobotic devices move to the targeted region. In one aspect, the time-lapsed images are taken periodically such as, e.g., about one image every 10 second, about one image every 1 second, about one image every 0.1 second, about one image every 0.001 second, about one image every 0.0001 second, about one image every 0.00001 second. In some cases, the time-lapsed images are taken in a range of 0.0001-10 second.

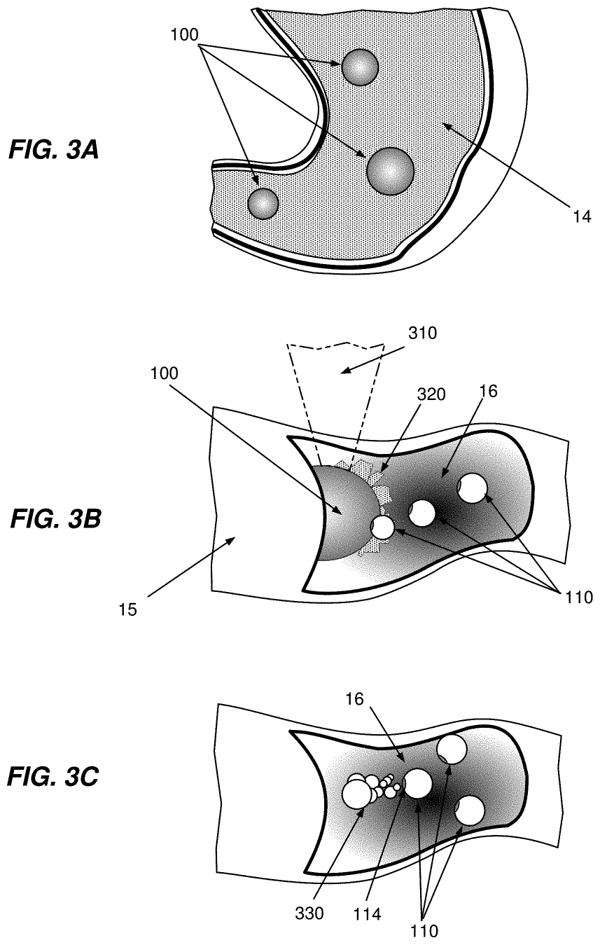

FIG. 3A is a schematic drawing of a portion of the stomach 14 of the mouse specimen 12 in FIG. 1, according to an implementation. The schematic drawing is at an instant in time after the ingestion of the three image-guided microrobotic devices 100 while the three image-guided microrobotic devices 100 are located in the stomach 14. The enteric coating of the microcapsules 120 (shown in FIG. 1) prevents their decomposition in the gastric fluids of the stomach 14.

FIG. 3B is a schematic drawing of a portion of the intestines 15 of the mouse specimen 12 in FIG. 1, according to an implementation. The schematic drawing is at an instant in time after the three image-guided microrobotic devices 100 have passed into the intestines 15 and are at or near a targeted region 16 having diseased tissue. At this time, a trigger device (e.g., the trigger device 420 in FIG. 4) of the image-guided microrobotic system 10 is generating a continuous-wave near infrared radiation (CW NIR) 320 that is directed to the targeted region 16 to induce phase transition and subsequent collapse/rupture on demand of the gelatin-based microcapsule 120 of the image-guided microrobotic device 100 at or near the target region 16, which unwraps the microcapsule 120 and activates the release, propulsion, and movement of three micromotors 110.

FIG. 3C is a schematic drawing of a portion of the intestines 15 of the mouse specimen 12 in FIG. 1, according to an implementation. The schematic drawing is at an instant in time after the continuous-wave near infrared radiation (CW NIR) 320 has caused the collapse/rupture the microcapsule 120 in FIG. 3B and the three micromotors 110 have been released. At this time, gas bubbles 330 are existing the open areas 114 of the three micromotors 110 causing active propulsion of the micromotors 120 into the diseased tissue of the targeted region 16. This active propulsion can promote retention and cargo delivery efficiency.

FIG. 4 is a simplified block diagram of components of an image-guided microrobotic system 400, according to implementations. The image-guided microrobotic system 400 includes an imaging subsystem 410 that takes one or more images of the specimen 25 to monitor movement of one or more image-guided microrobotic devices administered to the specimen 25. In one implementation, the imaging subsystem 410 is a PACT system such as, e.g., the PACT system 200 shown in FIG. 2. The imaging subsystem 410 is in communication with the specimen 25 to take images and in communication with the computing device 480 to communicate image data for the one or more images. The imaging subsystem 410 may be a component of the image-guided microrobotic system 400 or may be a separate component (as denoted by the dashed line). The specimen 25 may be located in communication with the components of the system 400 during operation. At other instances, the specimen 25 may be located elsewhere as denoted by the dashed line.

Returning to FIG. 4, the image-guided microrobotic system 400 also includes a trigger device 420 configured to provide activating energy to an area of the specimen 25. The activating energy is configured to cause the disintegration of the one or more microcapsules of the image-guided microrobotic device(s) administered to the specimen 25. In one aspect, the trigger device 420 is configured to cause the disintegration to occur in a length of time in a range from about 0.1 second to 1 second. In another aspect, the trigger device 420 is a high power radiation source (e.g., a 808 nm, 2 W continuous-wave (CW) near infrared (NIR) laser, 1064 nm pulsed near infrared Nd:YAG laser, or high-power CW fiber laser), which is configured to cause the disintegration to occur within 0.1 second. In another aspect, the trigger device 420 is a high-intensity focused ultrasound, which is configured to cause the disintegration to occur within 0.1-1 second. If the one or more microcapsules are gelatin-based and the imaging contrast layer is a material layer (e.g., an Au layer) that can convert the continuous-wave near infrared (CW-NIR) radiation to heat, the heat resulting from the CW-NIR radiation will cause in gel-sol phase transition of the gelatin-based one or more microcapsules to disintegrate them. Another example of a trigger device that can be used is a pulsed NIR laser (e.g., 660-1100 nm, 0.1-10 W, up to 10 cm penetration). Another example of a trigger device that can be used is a high-intensity focused ultrasound transmitter (0.5-30 MHz, 0.1-10 W, up to 20 cm penetration). Another example of a trigger device that can be used is a magnetic device.

The image-guided microrobotic system 400 includes a computing device 480 having one or more processors or other circuitry 482 and an internal non-transitory computer readable media (CRM) 484 in electrical communication with the processor(s) or other circuitry 782. The computing device 180 is also in electronic communication with the imaging subsystem 410 to send control signals and receive one or more photoacoustic images or data transmissions from the DAQ(s). The computing device 180 is also in electronic communication with the trigger device 410 to send control signals to activate the trigger device, e.g., when the computing device has executed instructions that determined that one or more image-guided microrobotic devices are at or near the target region. The processor(s) or other circuitry 482 of the computing system 480 of the image-guided microrobotic system 400 and, additionally or alternatively, other external processor(s) (e.g., a processor of the external computing system 489) can execute instructions stored on non-transitory computer readable media (e.g., internal non-transitory CRM 484 or optional external memory 492) to perform operations of the image-guided microrobotic system 400.

In certain implementations, an image-guided microrobotic system includes one or more processors and/or other circuitry that can execute instructions stored on a computer readable medium CRM to perform one or more operations of the image-guided microrobotic system and/or the imaging subsystem. In one aspect, the processor(s) and/or other circuitry and/or one or more external processors may execute instructions to perform: 1) determining and communicating control signals to system components, 2) performing algorithm(s) to reconstruct one or more images of the specimen acquire over time, e.g., reconstructing photoacoustic images from an acoustic signal received from the DAQ(s) of a photoacoustic imaging system such as the PACT system 200 in FIG. 2) evaluating one or more images of the specimen to determine when one or more image-guided microrobotic devices are near or at the targeted region based in part on one or more images of the specimen taken over time; and 4) communicating control signal to the trigger device to activate it when it is determined that the one or more image-guided microrobotic devices are at or near the target region based in part on the images constructed.

According to certain implementations, the computing system of an image-guided microrobotic system can perform parallel image processing. To perform parallel image processing, the computing device generally includes at least one processor (or “processing unit”). Examples of processors include, for example, one or more of a general purpose processor (CPU), an application-specific integrated circuit, an programmable logic device (PLD) such as a field-programmable gate array (FPGA), or a System-on-Chip (SoC) that includes one or more of a CPU, application-specific integrated circuit, PLD as well as a memory and various interfaces.

The computing system of an image-guided microrobotic system may be in communication with internal memory device and/or an external memory device. The internal memory device can include a non-volatile memory array for storing processor-executable code (or “instructions”) that is retrieved by one or more processors to perform various functions or operations described herein for carrying out various logic or other operations on the image data. The internal memory device also can store raw image data, processed image data, and/or other data. In some implementations, the internal memory device or a separate memory device can additionally or alternatively include a volatile memory array for temporarily storing code to be executed as well as image data to be processed, stored, or displayed. In some implementations, the computing system itself can include volatile and in some instances also non-volatile memory.

Returning to FIG. 4, optionally (denoted by dotted lines) the image-guided microrobotic system 400 includes a communication interface 485 and a display 486 in communication with the communication interface 485. The computing device 480 may be configured or configurable to output raw data, processed data such as image data, and/or other data over the communication interface 485 for display on the display 486. Optionally (denoted by dashed lines), the image-guided microrobotic system 400 may further include one or more of a communication interface 487 and an external computing system 489 in communication with the communication interface 487, a communication interface 490 and an external memory device 492 in communication with the communication interface 490 for optional storage of data to the external memory device 492, and/or a communication interface 493 in communication with a user interface 494 for receiving input from an operator of the image-guided microrobotic system 400. The optional user interface 494 is in electrical communication with the image-guided microrobotic system 400 through the communication interface 493 to be able to send a control signal to the computing device 480 based on input received at the user interface 494.

In some implementations, an image-guided microrobotic system includes a computing device configured or configurable (e.g., by a user) to: (i) output raw data, processed data such as image data, and/or other data over a communication interface to a display, (ii) output raw image data as well as processed image data and other processed data over a communication interface to an external computing device or system, (iii) output raw image data as well as processed image data and other data over a communication interface for storage in an external memory device or system, and/or (iv) output raw image data as well as processed image data over a network communication interface for communication over an external network (for example, a wired or wireless network). Indeed in some implementations, one or more of operations of an image-guided microrobotic system can be performed by an external computing device. The computing device may also include a network communication interface that can be used to receive information such as software or firmware updates or other data for download by the computing device. In some implementations, an image-guided microrobotic system further includes one or more other interfaces such as, for example, various Universal Serial Bus (USB) interfaces or other communication interfaces. Such additional interfaces can be used, for example, to connect various peripherals and input/output (I/O) devices such as a wired keyboard or mouse or to connect a dongle for use in wirelessly connecting various wireless-enabled peripherals. Such additional interfaces also can include serial interfaces such as, for example, an interface to connect to a ribbon cable. It should also be appreciated that one or more of components of the iSVS system can be electrically coupled to communicate with the computing device over one or more of a variety of suitable interfaces and cables such as, for example, USB interfaces and cables, ribbon cables, Ethernet cables, among other suitable interfaces and cables.

The described electrical communication between components of an image-guided microrobotic systems may be able to provide power and/or communicate data. The electrical communication between components of the image-guided microrobotic systems described herein may be in wired form and/or wireless form.

### III. Image-Guided Microrobotic Methods

A. Methods of Fabricating Image-Guided Microrobotic Devices

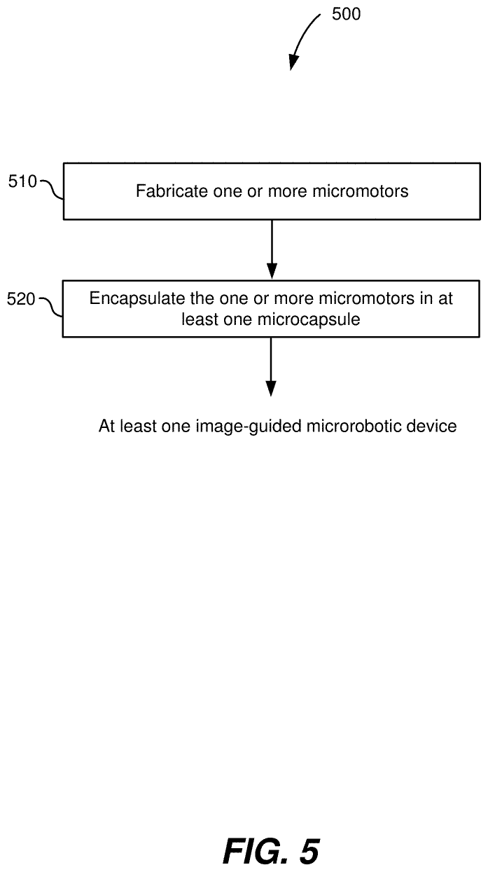

Methods of fabricating at least one image-guided microrobotic device includes at least two operations: 1) fabricating one or more micromotors; and 2) encapsulating the one or more micromotors in at least one microcapsule. FIG. 5 depicts a flowchart 500 illustrating operations of a method of fabricating at least one image-guided microrobotic device, according to implementations. At operation 510, one or more micromotors are fabricated. In one aspect, the micromotors are constructed using an embedding method. At operation 520, the one or more micromotors are encapsulated in at least one microcapsule. In one aspect, the at least one image-guided microrobotic device and contents are ingestible.

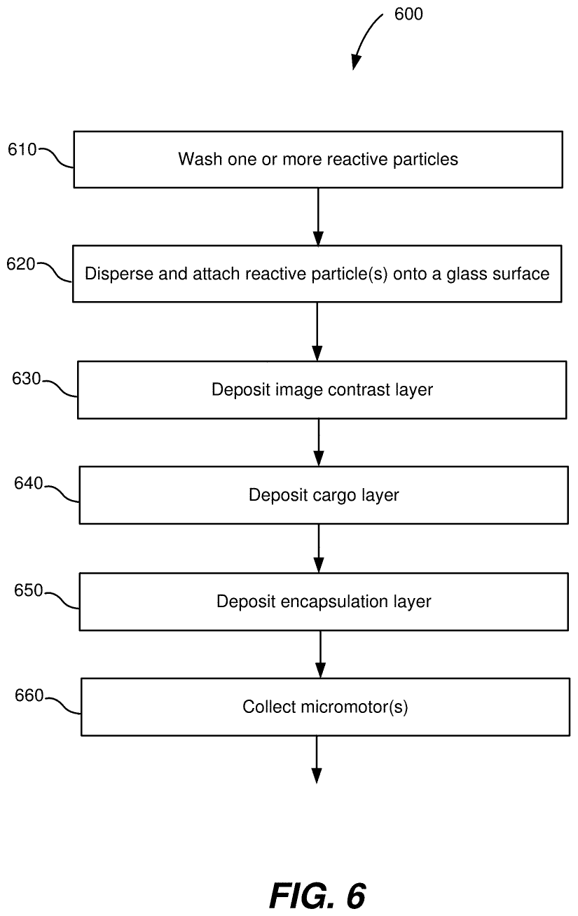

FIG. 6 depicts a flowchart 600 with an example of sub-operations of operation 510 in FIG. 5, according to one aspect. At operation 610, one or more reactive particles (e.g., Mg particles having a diameter of about 20 μm) are washed with acetone and dried at room temperature. In some cases, the one or more particles are washed multiple times such as three times. At operation 620, the one or more reactive particles are dispersed in acetone and attached to glass slide(s), e.g., using physical adsorption. For example, Mg particles having a diameter of about 20 μm may be dispersed in acetone with a particle concentration of ˜0.1 g mL⁻¹ and then spread on the glass slide(s) at room temperature. After the acetone evaporated, Mg particles were attached onto the surface of the glass slides through physical adsorption, exposing the majority of the surface areas of the particles to air. At operation 630, an imaging contrast layer is deposited over the glass slide(s) coated with the reactive particles. Some deposition techniques that can be used include E-beam, glancing angle deposition, sputter thin film deposition, and atomic layer deposition. In one example, the glass slides coated with Mg particles were deposited with an Au layer (e.g., about 100 nm in thickness) using an electron-beam evaporator. An example of a commercially-available electron-beam evaporator is the Mark 40 electron-beam evaporator sold by CHA Industries. An Au layer may facilitate the autonomous chemical propulsion in gastrointestinal fluids and enhances photoacoustic contrast of micromotors. At operation 640, a cargo layer is deposited over the glass slide(s) coated with the reactive particles. In one aspect, a mixture containing alginate (2%, w/v) and doxorubicin is dropped on the glass slide(s) and then dried with N₂ gas. Then, aqueous CaCl₂ (0.2 mL of 5%, w/v) is dropped onto the glass slide(s) to cross-link alginate, and after 30 minutes, the glass slide(s) are washed with pure water and dried with N₂ gas. At operation 650, an encapsulation layer is deposited over the glass slide(s) coated with the reactive particles. In one example, the glass slide(s) is coated with a parylene C layer (e.g., 750 nm in thickness) using a parylene coater. An example of a commercially-available parylene coater is the Labtop 3000 coater made by Curtiss-Wright. At operation 660, the one or more micromotors are collected from the slide(s). In one example, the one or more micromotors are collected by scratching them from the glass slide(s).

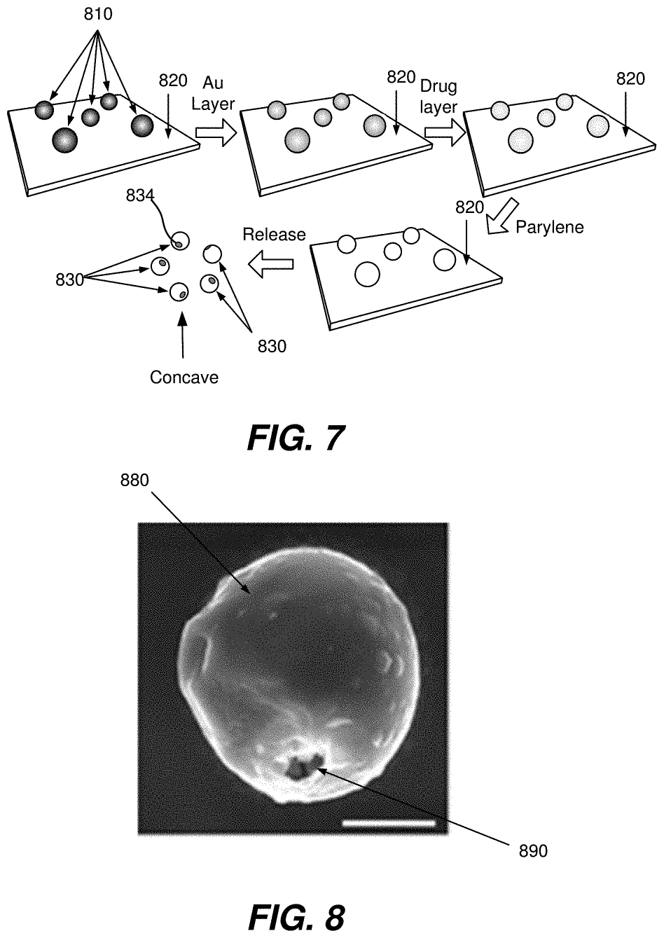

FIG. 7 is schematic drawing of operations of a fabrication flow for ingestible Mg-based micromotors, according to an implementation. In the first operation, Mg microparticles 810 with diameters of about 20 μm were dispersed onto the glass slides 820. At the second operation, a gold layer is deposited over the glass slides 820. The gold layer may facilitate the autonomous chemical propulsion in gastrointestinal fluids and enhances photoacoustic contrast of the micromotors. At the third operation, an alginate hydrogel layer is deposited onto the slides by dropping aqueous solution containing alginate and drugs (e.g., doxorubicin) on the slides 820. At the fourth operation, a parylene layer, acting as a shell scaffold that ensures the stability during propulsion, is deposited onto the slides 820. At the fifth operation, the micromotors are released from the slides. As shown, an open area 834 on the side of the micromotors that was in contact with the surface of the slides does not have any coating over the Mg microparticles. The open area 834 is concave.

FIG. 8 is a scanning electron microscope image of an ingestible Mg-based spheroid micromotor 880 fabricated using the operations described in FIG. 7, according to an implementation. The scanning electron microscope image was taken by a field emission scanning electron microscope made by Sirion at an operating voltage of 10 keV. To improve conductive for imaging, the sample was coated with a 5-nm carbon layer using a commercially available EM ACE600 Carbon Evaporator made by Leica. As shown in FIG. 8, the ingestible Mg-based spherical micromotor 880 includes a small opening 890 of about 2 μm in diameter. The small opening 890 is attributed to the surface contact of the Mg microparticles with the glass slides during various layer coating steps. The small opening 890, acts as a catalytic interface for gas propulsion in the intestinal environment.

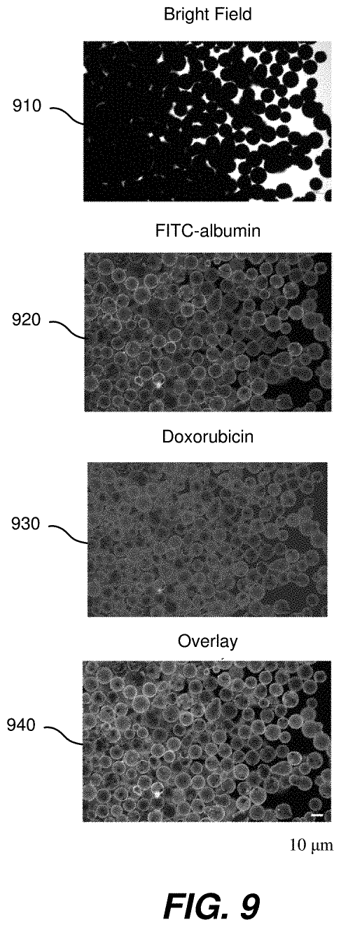

FIG. 9 depicts bright field and fluorescence microscopic images of the ingestible Mg-based micromotors fabricated using the operations described in FIG. 7, according to an implementation. The bright field and fluorescence microscopic images of the ingestible Mg-based micromotors were taken with a Zeiss AXIO optical microscope. At the top is a bright field image 910 of the ingestible Mg-based micromotors. To observe the structure of the DOX-loaded (i.e. loaded with Doxorubicin) micromotors using fluorescence imaging, the ingestible Mg-based micromotors were stained with FITC-albumin. The second image 920 is a gray-scaled version of the green fluorescence image of the ingestible Mg-based micromotors stained with FITC-albumin. Labeling of FITC-albumin onto the ingestible Mg-based micromotors was carried out by dip-coating the micromotors-loaded glass slides in a 0.2 mL of FITC-albumin solution (0.2 mg mL⁻¹), followed by dip-coating in an alginate solution (2%, w/v). The third image 930 is a gray-scaled version of the red fluorescence image from doxorubicin (DOX) of the ingestible Mg-based micromotors. The fourth image 940 is a gray-scaled version of a color overlay of green and red. The images confirm the successful drug loading of the ingestible Mg-based micromotors. The red fluorescence in the micromotors are from DOX channel, which indicates successful loading.

Returning to operation 520 in the flowchart 500 shown in FIG. 5, in one example, the micromotor(s) are encapsulated into enteric gelatin microcapsules by the controlled emulsion technique discussed in Yin, N., et al. “Agarose particle-templated porous bacterial cellulose and its application in cartilage growth in vitro,” Acta Biomater. 12, 129-138 (2015) and Li, J., et al., “Enteric micromotor can selectively position and spontaneously propel in the gastrointestinal tract,” ACS Nano 10, 9536-9542 (2016), which are hereby incorporated by reference in their entireties. Other examples of techniques that can be used include molecular assembly, 3D printing, and polymerization reaction.

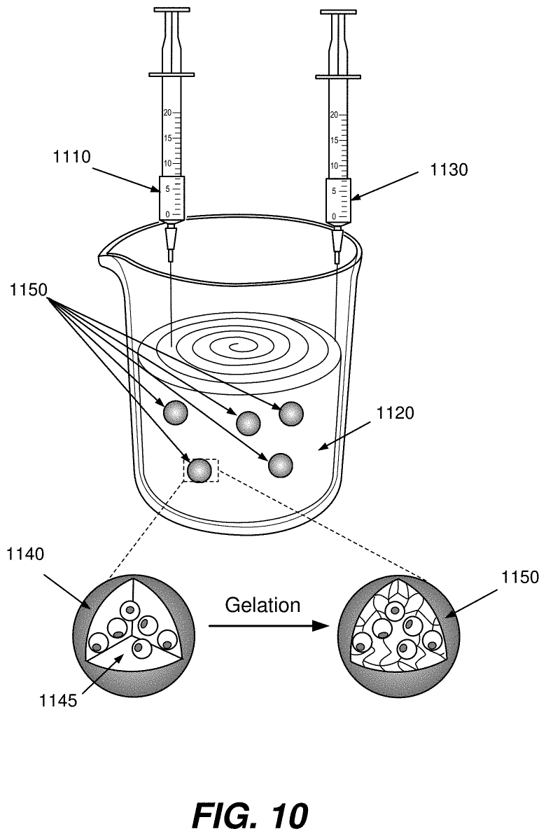

FIG. 10 is a schematic drawing depicting operations in an exemplary operation using the controlled emulsion technique for encapsulating micromotors in enteric gelatin microcapsules, according to an implementation. A gelatin micromotor mixture 1110 is added to paraffin liquid 1120. In one example, the gelatin micromotor mixture 1110 contains gelatin (5%, w/v) and micromotors (5%, w/v) at 40-60° C. may be extruded from a 30-gauge needle into 50 mL of liquid paraffin at about 60° C. Pure water may be used as the solvent, in which micromotors remained stable due to the formation of a compact hydroxide passivation layer on the Mg surfaces. Subsequently, an enteric polymer solution 1130 is extruded into the paraffin liquid 1120. In one example, the enteric polymer solution 1130 consists of 100 mg of Eudragit L-100 in 2 mL organic solvent mixture (acetone:methanol=1:1, v/v) as discussed in Chourasia, M. K., Jain, S. K., “Design and development of multiparticulate system for targeted drug delivery to colon,” Drug Deliv. 11, 201-207 (2004), which is hereby incorporated by reference in its entirety. The extruded solution is allowed to sit to allow liquid to evaporate and then temperature lowered. In one example, the extruded solution is kept at about 60° C. for 4 hours to evaporate the acetone and methanol, and then the temperature was lowered to about 0° C. with an ice bath. In order to harvest the image-guided microrobotic devices 1150, cold water is added to the paraffin liquid 1120 and then stirring is performed to separate the image-guided microrobotic devices 1150 from the paraffin liquid 1020 into the water. Gelation of the droplets 1140 with the micromotors 1145 occurs forming the image-guided microrobotic devices 1150. In one example, cold water at about 4° C. is added into the liquid paraffin with magnetic stirring for more than about 20 minutes, and most image-guided microrobotic devices 1150 separate from the liquid paraffin 1120 into the water. The water containing image-guided microrobotic devices 1150 is then extracted and washed with hexane, e.g., three times. The size of the image-guided microrobotic devices 1150 can be controlled by varying the rotational speed of magnetic stirring between 100 and 1000 rpm. The collected image-guided microrobotic devices 1150 are rinsed with an aqueous hydrochloric acid solution (pH=2) and then washed with pure water to remove the hydrochloric acid. Subsequently, the image-guided microrobotic devices 1150 are cross-linked through incubation with glutaraldehyde for 1 hour followed by water rinse.

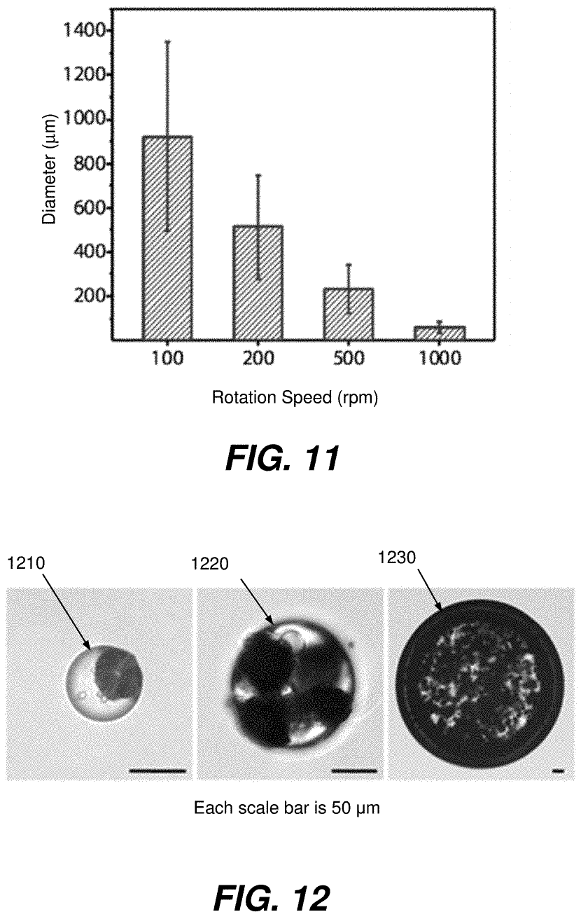

In one aspect, the image-guided microrobotic devices can be fabricated to have a particular approximate diameter by setting the rotational speed of the magnetic stirring. FIG. 11 depicts a bar graph with rotational speeds of magnetic stirring for different diameters of image-guided microrobotic devices, according to an implementation. The error bars represent standard deviations. In one aspect, a rotational speed of 100 rpm corresponds to an approximate diameter of about 900 μm, a rotational speed of 200 rpm corresponds to an approximate diameter of about 500 μm, a rotational speed of 500 rpm corresponds to an approximate diameter of about 250 μm, and a rotational speed of 1000 rpm corresponds to an approximate diameter of about 50 μm. FIG. 12 depicts microscopic images of three image-guided microrobotic devices 1210, 1220, 1230, with diameters of 68 μm, 136 μm, and 750 μm respectively as formed by magnetic stirring at rotational speeds of 1000 rpm, 500 rpm, and 200 rpm respectively, according to an implementation.

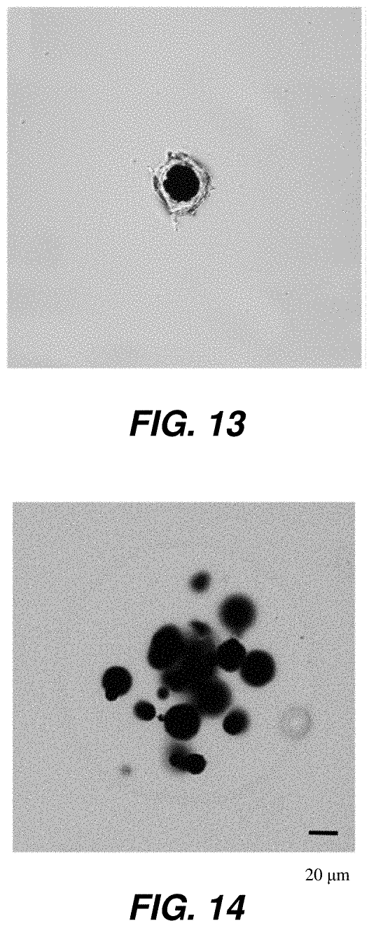

FIG. 13 and FIG. 14 depicts bright field images of ingestible image-guided microrobotic devices with Mg-based micromotors fabricated using the operations described with reference to FIG. 7 and encapsulated using the operations described with reference to FIG. 10, according to an implementation. These bright field images show ingestible image-guided microrobotic devices that are formed with different sizes by employing different rotational speeds of magnetic stirring.

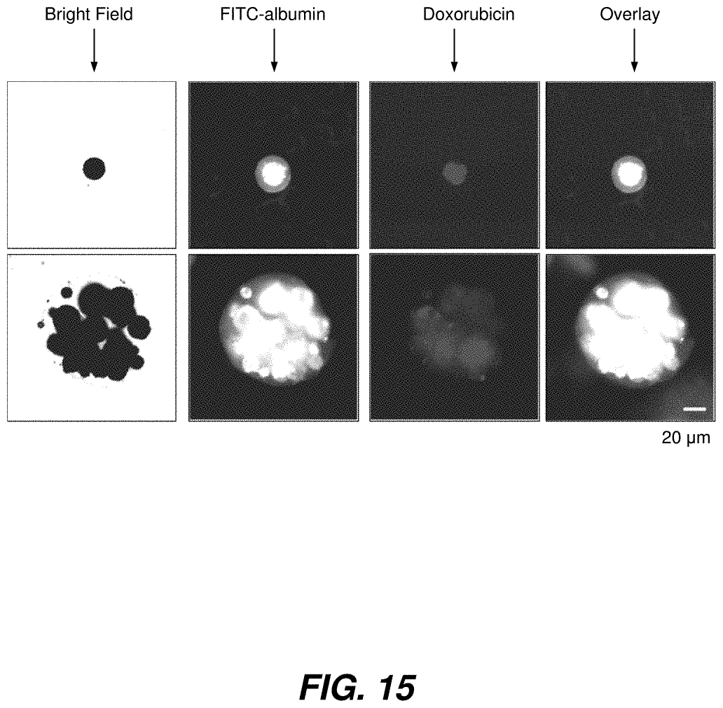

FIG. 15 depicts bright field and fluorescence microscopic images of the ingestible Mg-based micromotors fabricated using the operations described in FIG. 7, according to an implementation. The bottom row of images is taken at a higher magnification than the lower row of images. The bright field and fluorescence microscopic images of the ingestible Mg-based micromotors were taken with a Zeiss AXIO optical microscope. The pair of images in the leftmost column are bright field images of the ingestible Mg-based micromotors at different magnifications. To observe the structure of the DOX-loaded (i.e. loaded with Doxorubicin) micromotors using fluorescence imaging, the ingestible Mg-based micromotors were stained with FITC-albumin. The next (second) column of images are gray-scaled versions of the green fluorescence images of the ingestible Mg-based micromotors stained with FITC-albumin. Labeling of FITC-albumin onto the ingestible Mg-based micromotors was carried out by dip-coating the micromotors-loaded glass slides in a 0.2 mL of FITC-albumin solution (0.2 mg mL⁻¹), followed by dip-coating in an alginate solution (2%, w/v). The next (third) column of images are gray-scaled versions of the red fluorescence image from doxorubicin (DOX) of the ingestible Mg-based micromotors. The right column of images are gray-scaled versions of a color overlay of green and red. The images confirm the successful drug loading of the ingestible Mg-based micromotors. The red fluorescence is from the DOX channel.

Performance

For deep tissue imaging in vivo, image-guided microrobotic devices according to one aspect have higher optical absorption than the blood in the specimen. Using a PACT system, the photoacoustic performance of ingestible Mg-based image-guided microrobotic devices fabricated using the operations described in FIG. 7 was evaluated. The PACT system employed is described in Li, L., et al., “Single-impulse panoramic photoacoustic computed tomography of small-animal whole-body dynamics at high spatiotemporal resolution,” Nat. Biomed. Eng. 1, 0071 (2017), which is hereby incorporated by reference in its entirety. For the evaluation, the image-guided microrobotic devices, the bare Mg microparticles, and blood were separately injected into three silicone tubes. Both ends of the tubes were sealed with agarose gel (2%, w/v). the PACT system employed a 512-element full-ring ultrasonic transducer array (e.g., the Imasonic SAS with 50 mm ring radius, 5.5 MHz central frequency, and more than 90% one-way bandwidth) for 2D panoramic acoustic detection. Each transducer element had a cylindrical focus, 0.2 NA, 20 mm element elevation size, 0.61 mm pitch, and 0.1 mm inter-element spacing in the array. A 512-channel preamplifier (26 dB gain) was directly connected to the ultrasonic transducer array housing, minimizing cable noise. The pre-amplified photoacoustic signals were digitized using a 512-channel data acquisition system (e.g., a system including four SonixDAQs, Ultrasonix Medical ULC; 128 channels each; 40 MHz sampling rate; 12-bit dynamic range) with programmable gain up to 51 dB. The digitized radio frequency data was first stored in the onboard buffer, then transferred to a computing device and reconstructed using the dual-speed-of-sound half-time universal back-projection algorithm as described in Li, L., et al., “Single-impulse panoramic photoacoustic computed tomography of small-animal whole-body dynamics at high spatiotemporal resolution,” Nat. Biomed. Eng. 1, 0071 (2017), which is hereby incorporated by reference in its entirety. Near infrared light experiences the least attenuation in mammalian tissues, permitting the deepest optical penetration.

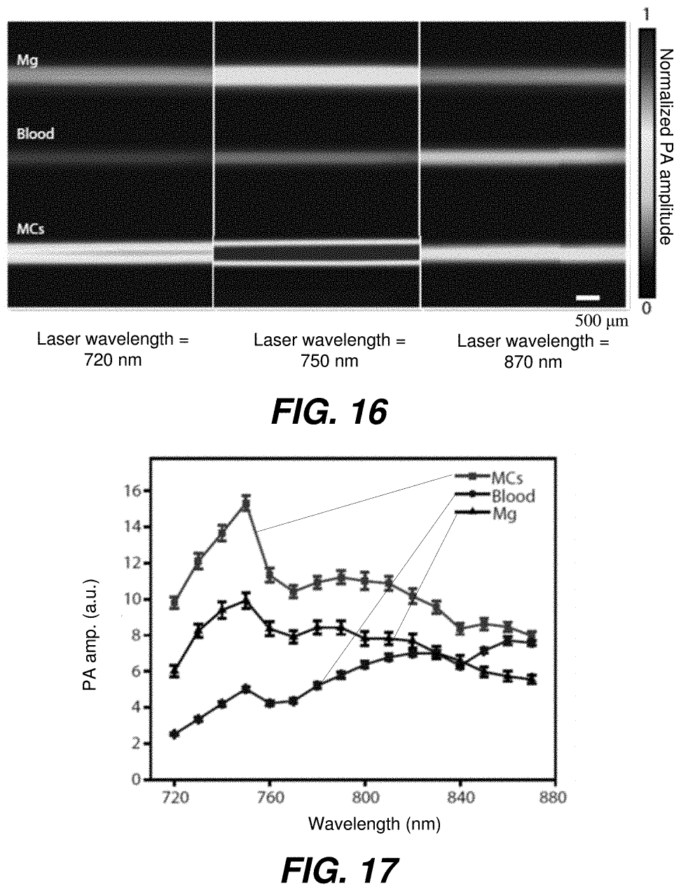

FIG. 16 are PACT images of bare Mg particles, whole blood, and the ingestible Mg-based image-guided microrobotic devices in three silicone rubber tubes using laser wavelengths at 720, 750, and 870 nm, respectively, according to an implementation. As shown, the image-guided microrobotic devices exhibit strong photoacoustic contrast in the near infrared wavelength region, ranging from 720 to 890 nm. In order to assess quantitatively the optical absorption of the image-guided microrobotic devices, amplitude values were extracted from the photoacoustic images in FIG. 16 and subsequently calibrated with the optical absorption of hemoglobin as provided in De la Zerda, A., et al. “Family of enhanced photoacoustic imaging agents for high-sensitivity and multiplexing studies in living mice,” ACS Nano 6, 4694-4701 (2012) and Eghtedari, M., et al., “High sensitivity of in vivo detection of gold nanorods using a laser optoacoustic imaging system,” Nano Lett. 7, 1914-1918 (2007), which are hereby incorporated by reference in their entireties.

FIG. 17 is a graph with plots of the measured PACT photoacoustic spectra of the ingestible Mg-based image-guided microrobotic devices, whole blood, and Mg particles, respectively. At the wavelength of 750 nm, the image-guided microrobotic devices display the highest photoacoustic amplitude of 15.3. The bare Mg particles display a similar photoacoustic spectrum, with a lower photoacoustic peak with an amplitude of 10.0 at 750 nm. Adding an Au layer in the micromotors improves the imaging sensitivity in the near infrared wavelength region. An example of materials that increase imaging sensitivity in other types of imaging can be found in Guo, W., et al., “CsxWO₃ nanorods coated with polyelectrolyte multilayers as a multifunctional nanomaterial for bimodal imaging-guided photothermal/photodynamic cancer treatment,” Adv. Mater. 29, 1604157 (2017) and Ji, T., Lirtsman, V. G., Avny, Y., Davidov, D., “Preparation, characterization, and application of Au-shell/polystyrene beads and Au-shell/magnetic beads,” Adv. Mater. 13, 1253-1256 (2001), which are hereby incorporated by reference in their entireties. As shown in FIG. 17, there is approximately a three-fold increase in photoacoustic amplitudes of the image-guided microrobotic devices as compared to that of the whole blood at 750-nm. This 3-fold increase in provides sufficient contrast to detect the image-guided microrobotic devices in vivo using 750-nm illumination.

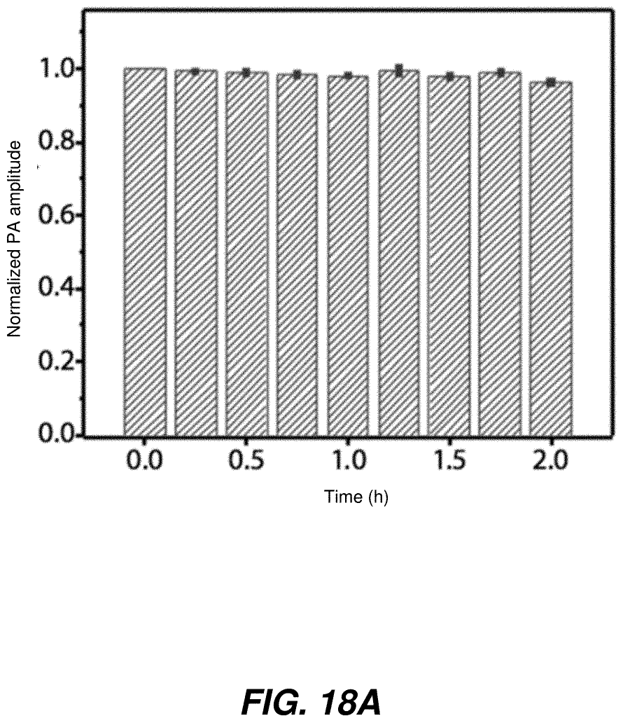

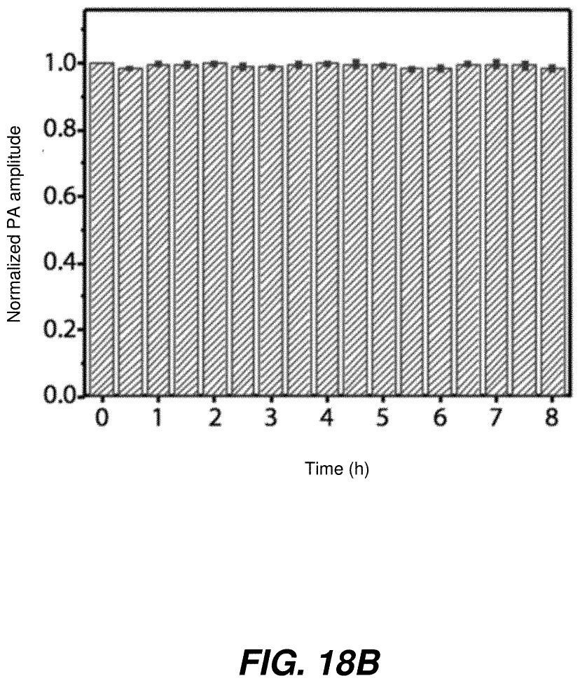

To evaluate the stability of the image-guided microrobotic devices under pulsed NIR photoacoustic excitation, the PA signal fluctuation of the image-guided microrobotic devices was measured during photoacoustic imaging using PACT. FIG. 18A is a bar graph of normalized PA amplitude over time under NIR illumination used in PACT in vitro, according to an implementation. FIG. 18B is a bar graph of normalized PA amplitude over time under NIR illumination used in PACT in vivo, according to an implementation. The negligible changes in the PA signal amplitude during the operation suggest a remarkably high photostability of the image-guided microrobotic devices.

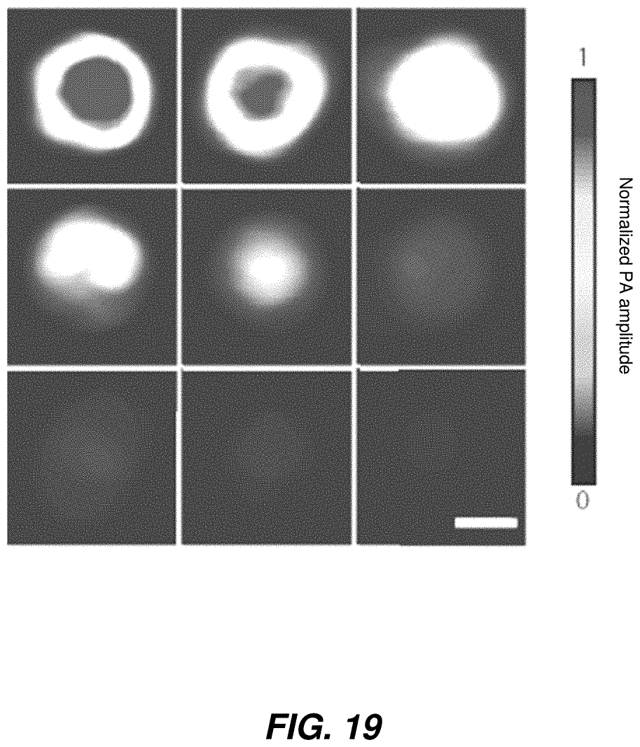

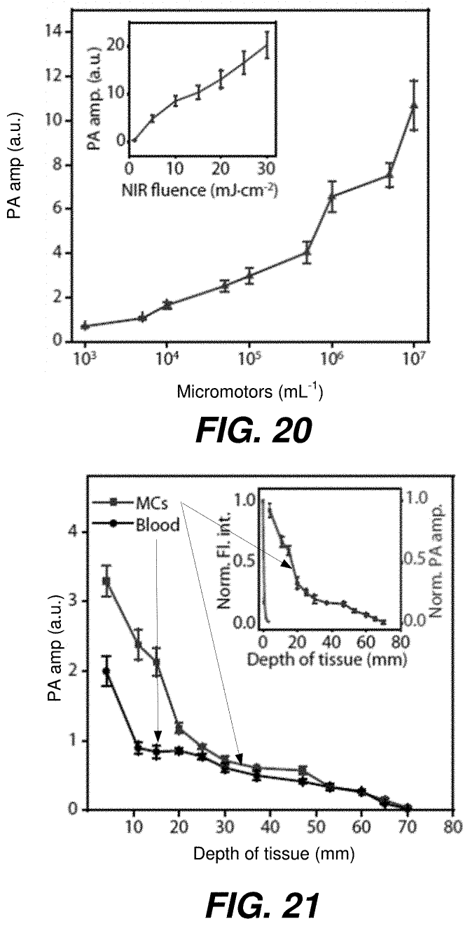

FIG. 19 depict nine (9) gray-scaled versions of photoacoustic images using PACT for different increasing concentrations (loading amounts) of micromotors, according to an implementation. FIG. 20 depicts a graph with a plot of the PA amplitude vs. concentrations (loading amounts) of micromotors and PA amplitude vs. fluence level of NIR illumination, according to an implementation. FIGS. 19 and 20 shown the photoacoustic images and the corresponding photoacoustic amplitudes of single image-guided microrobotic devices with different concentrations of micromotors. As expected, the PA amplitude of the micromotors almost linearly increases with the NIR light fluence as shown in inset plot of FIG. 20.

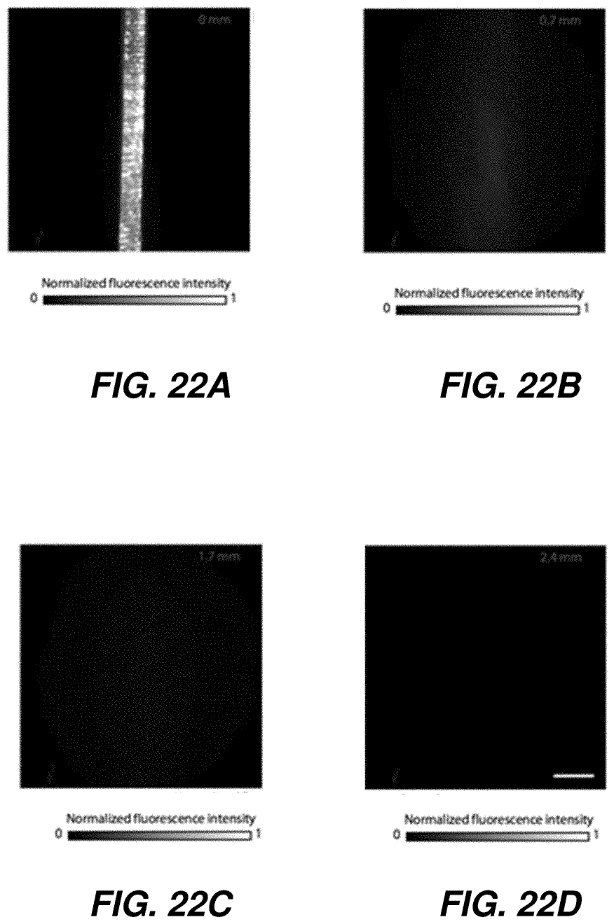

Maximum detectable depth of image-guided microrobotic devices using PACT was evaluated. FIG. 21 depicts a graph with a plot of the PA amplitude vs. depth of tissue for both image-guided microrobotic devices and blood and a plot of normalized fluorescence intensity vs. depth of tissue for both image-guided microrobotic devices and blood, according to an implementation. The PA amplitude was determined using PACT. FIG. 22A is a fluorescence image of the image-guided microrobotic devices in a silicone tube under chicken breast tissues with a thickness of 0 mm, according to an implementation. FIG. 22B is a fluorescence image of the image-guided microrobotic devices in a silicone tube under chicken breast tissues with a thickness of 0.7 mm, according to an implementation. FIG. 22C is a fluorescence image of the image-guided microrobotic devices in a silicone tube under chicken breast tissues with a thickness of 1.7 mm, according to an implementation. FIG. 22D is a fluorescence image of the image-guided microrobotic devices in a silicone tube under chicken breast tissues with a thickness of 2.4 mm, according to an implementation. The micromotors showed dramatically decreased fluorescence intensity when covered by thin tissues (0.7-2.4 mm in thickness) and became undetectable quickly as shown FIGS. 22A-D. By contrast, PACT can image the micromotors inside tissue as deep as about 7 cm as shown in FIG. 21, which reveals that the key advantage of PACT lies in the high spatial resolution and high molecular contrast for deep imaging in tissues as discussed in Li, L. et al., “Single-impulse panoramic photoacoustic computed tomography of small-animal whole-body dynamics at high spatiotemporal resolution,” Nat. Biomed. Eng. 1, 0071 (2017), which is hereby incorporated by reference in its entirety.

B. Methods of Navigating and Activating Image-Guided Microrobotic Devices

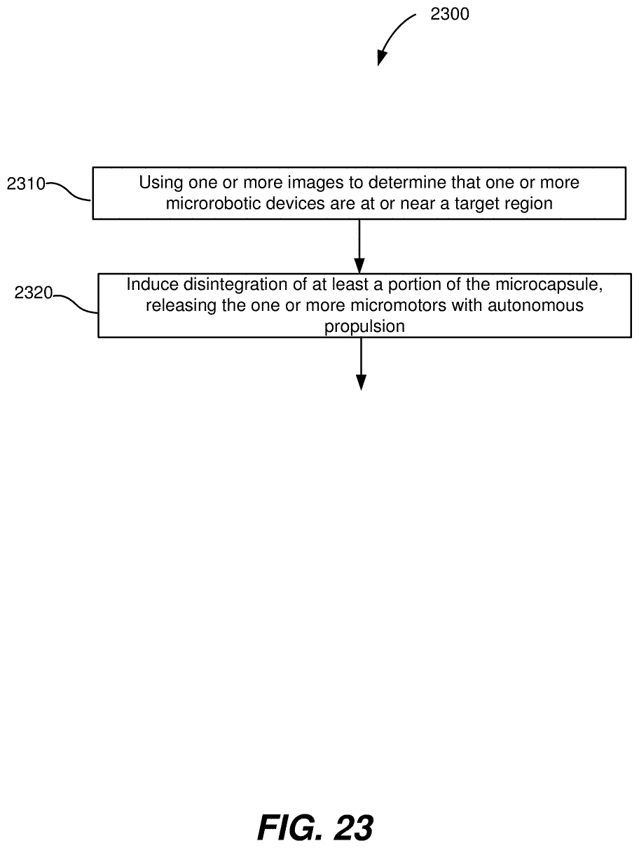

FIG. 23 depicts a flowchart 2300 illustrating operations of an image-guided microrobotic method, according to implementations. At operation 2310, one or more images are used to determine that one or more image-guided microrobotic devices are at or near a target region. The one or more images may be time-lapsed images taken periodically over time. In operation 510, the computing device determines from the one or more images directly or from input from an operator that the that one or more image-guided microrobotic devices are at or near a target region. For example, the one or more images may be analyzed to determine when the image-guided microrobotic devices are at or near the targeted region. Microrobots and the targeted regions will be recognized by their shapes, amplitude, frequency or other characteristic information, or they can be recognized via machine learning. Once both recognized, the motion of the microrobots will be tracked and decision will be made when microrobots reach the target location.

Each image-guided microrobotic device includes a partial coating disposed over a reactive particle. The partial coating includes at least one area that exposes the reactive particle to ingress by fluid that can cause a reaction that releases gases that when exiting the at least one area cause autonomous propulsion. The partial coating may include an imaging contrast agent and cargo such as, e.g., therapeutic drugs. The one or more images may be generated by an imaging subsystem or a separate imaging system that employs, e.g., PACT, ultrasound, magnetic resonance imaging, X-ray CT, PET, DOT, PAM, OCT.

In one implementation, PACT is used such as by employing the PACT system 200 shown in FIG. 2. The photoacoustic signals received by the DAQ(s) are low-pass filtered with cut-off frequencies determined by the maximum distance from a point in the specimen being imaged to the transducer elements. Using the PACT system 200, the pre-amplified photoacoustic signals are digitized using a 512-channel data acquisition system (DAQ). The digitized radio frequency data is first stored in the onboard buffer, then transferred to a computing device and reconstructed using the dual-speed-of-sound half-time universal back-projection algorithm

Returning to FIG. 23, at operation 2320, disintegration of at least a portion of one or more of the microcapsules is induced. The disintegration is induced by one or more triggering mechanisms such as photothermal effect, acoustic thermal effect, acoustic radiation force, magnetic force. In one implementation, the triggering mechanism is a continuous wave (CW) near-infrared (NIR) light irradiation. If the microcapsule is gelatin-based and the imaging contrast layer is an Au layer, the Au layer can convert the NIR light to heat resulting in gel-sol phase transition of the gelatin-based microcapsule to disintegrate the microcapsule.

### IV. Results

In Vitro Evaluation

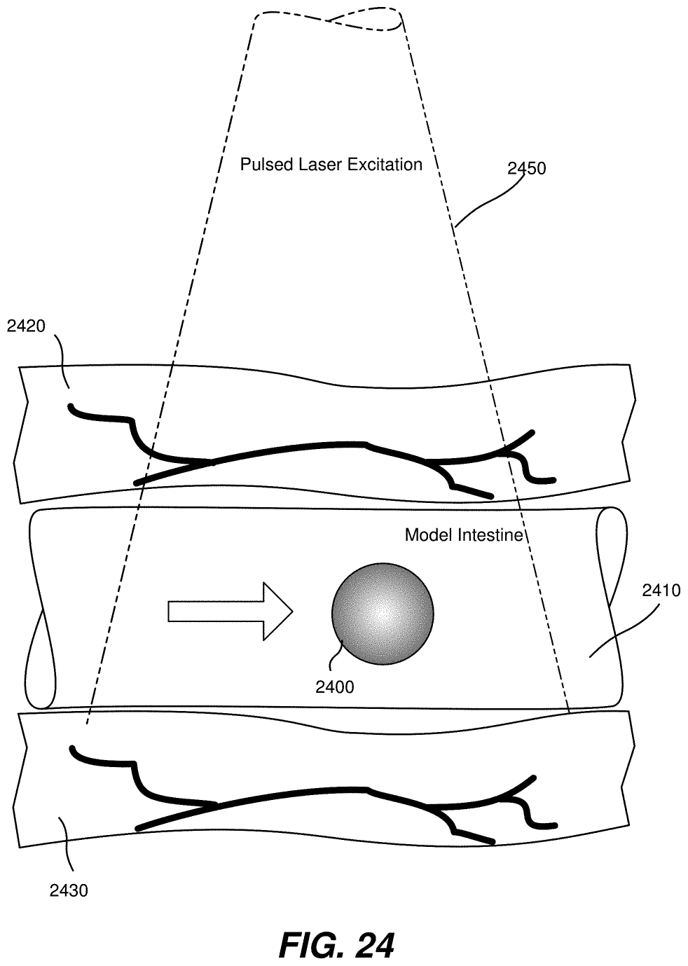

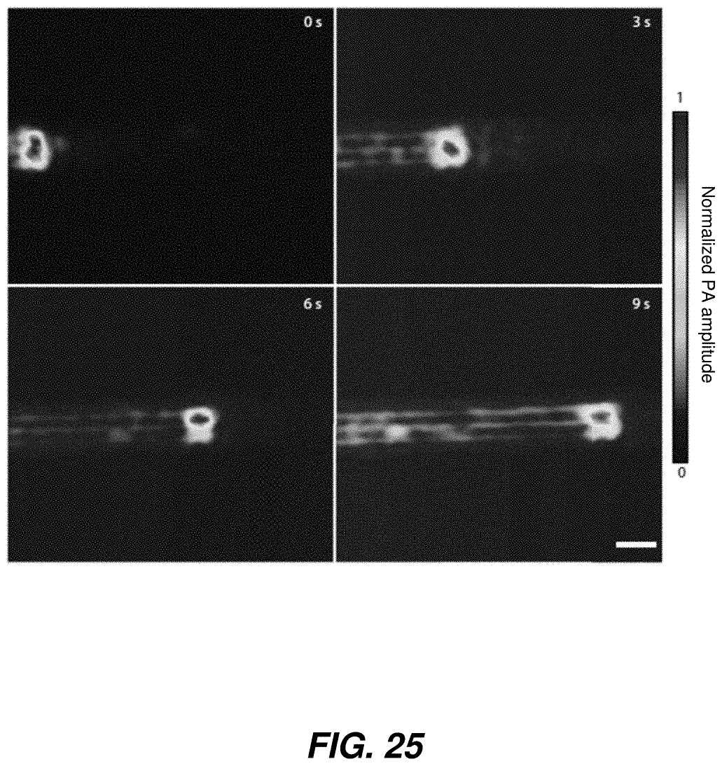

To evaluate the dynamics of image-guided microrobotic techniques of certain implementations, photoacoustic imaging experiments were conducted in vitro, where silicone rubber tubes (e.g., silicone rubber tubes with an inner diameter of 0.5 mm sold by Dow Silicones) modeled intestines. FIG. 24 is a schematic drawing of a silicon rubber tube modeled intestine 2410 sandwiched between two portions of chicken breast tissue 2420, 2430, according to an implementation. An image-guided microrobotic device 2400 has been injected into the silicon rubber tube modeled intestine 2410. Migration of the image-guided microrobotic device 2400 was driven by microfluidic pumping. A PACT system uses pulsed laser excitation irradiation 2450 to illuminate the tissues. The thickness of the tissue above the image-guided microrobotic device 2400 is 10 mm. The PACT system takes time-lapsed photoacoustic images to illustrate real-time tracking of the migration of the image-guided microrobotic device 2400 in the silicon rubber tube modeled intestine 2410. FIG. 25 depicts four time-lapsed photoacoustic images of the normalized photoacoustic amplitude taken by the PACT system at time=0 s, 3 s, 6 s, and 9 s, according to an implementation. The four time-lapsed photoacoustic images show the real-time tracking of the migration of the injected image-guided microrobotic device 2400 through the silicon rubber tube modeled intestine 2410 during the time period of time=0 s to time=9 s.

Triggering Collapse of Microcapsule(s)

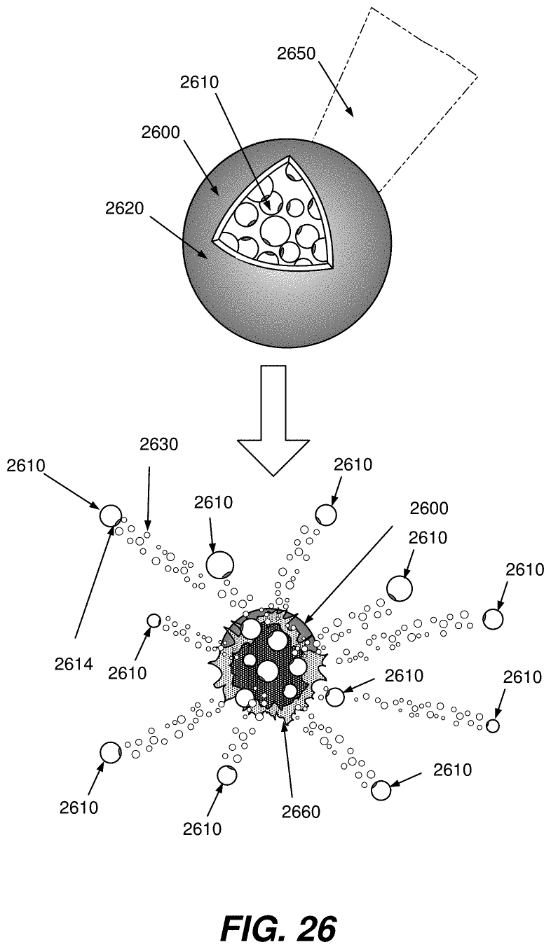

In addition to tracking image-guided microrobotic devices, propulsion of the micromotors upon unwrapping from the microcapsules can be activated on demand by applying high power CW NIR irradiation and/or other triggering mechanisms. FIG. 26 is a schematic drawing illustrating activation on demand of propulsion of micromotors 2610 upon unwrapping from a microcapsule 2620 activated by high power CW NIR irradiation 2650 directed at a region with the image-guided microrobotic device 2600, according to an implementation. When intact, the image-guided microrobotic device 2600 includes a plurality of micromotors 2610 and a gelatin-based microcapsule 2620, each micromotor 2610 has an Au layer. Upon application of the high power CW NIR irradiation 2650 to the image-guided microrobotic device 2600, the Au layer of the micromotors 2610 can effectively convert the NIR light to heat, resulting in a gel-sol phase transition of the gelatin-based microcapsule 2620 and collapse 2660 of the gelatin-based microcapsule 2620, followed by release of the micromotors 2610. In one aspect, the CW NIR-triggered disintegration of the microcapsule 2620 occurs within 0.1 s. Such a photothermal effect also significantly accelerates the Mg-water chemical reactions at the reactive Mg particle and thus enhances the chemical propulsion of the micromotors 2610.

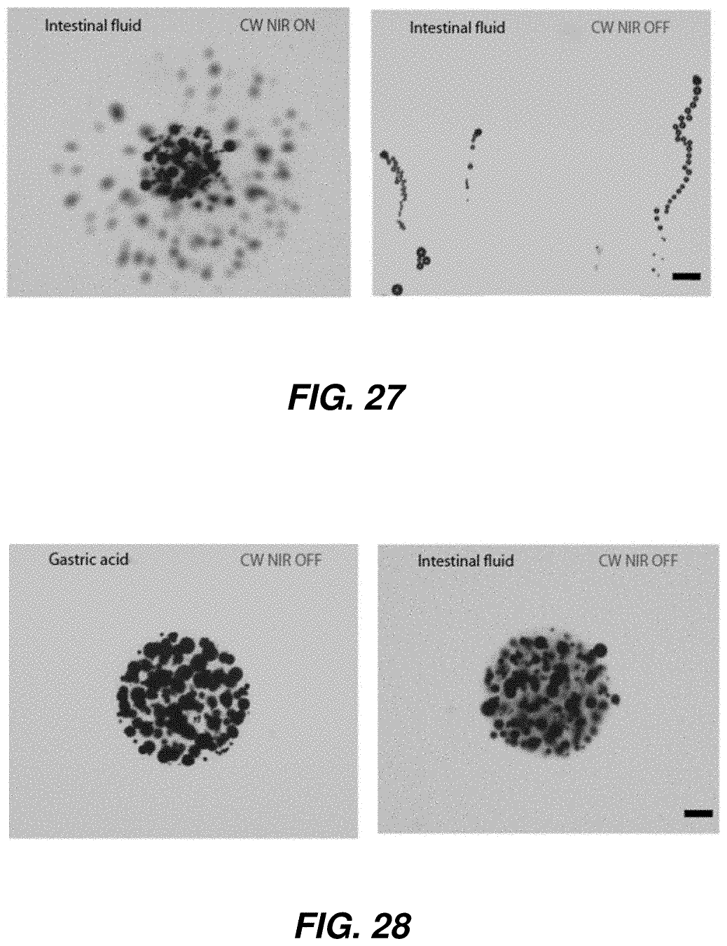

In one implementation, CW NIR-activated propulsion of the micromotors is employed. To evaluate the CW NIR-activated propulsion, a PBS solution of 30 μL mixed with image-guided microrobotic devices was dropped on a piece of gene frame and a glass coverslip was placed over the gene frame. A CW NIR laser (e.g., 808 nm, 2 W CW NIR laser with a focal diameter of about 0.8 cm) was used to irradiate the image-guided microrobotic devices obliquely (e.g., at an angle of 45 degree) with the light beam aligned to the focus of a microscope. The image-guided microrobotic devices were irradiated before they completely sank to the bottom of the glass slide. The disintegration of the image-guided microrobotic devices occurred within 0.1 s exposure of the CW NIR light. In addition, during each respiration cycle, the resting time (the duration free of respiration motion) is typically longer than 0.3 s as discussed in Li, L., et al., “Single-impulse panoramic photoacoustic computed tomography of small-animal whole-body dynamics at high spatiotemporal resolution,” Nat. Biomed. Eng. 1, 0071 (2017). Thus, once the real-time PACT detects that image-guided microrobotic devices have reached the targeted area, the CW NIR light can trigger the release during the resting time, avoiding the influence of respiration motion. The process of the NIR-triggered disintegration of the image-guided microrobotic devices and the propulsion of the micromotors was captured using a high-speed camera (e.g., Axiocam 720 mono) at 100 and 25 frames s⁻¹, respectively. FIG. 27 includes two time-lapsed microscopic images showing the use of high power CW NIR irradiation to trigger the collapse of the microcapsule of an image-guided microrobotic device and the activation/propulsion of the micromotors, according to an implementation. In the left microscopic image, the CW NIR irradiation has triggered the collapse of the microcapsule of an image-guided microrobotic devices allowing the micromotors to be released. In the right microscopic image, which occurs after the time of the left image, propulsion has caused the micromotors to move away from the region of the collapsing microcapsule.

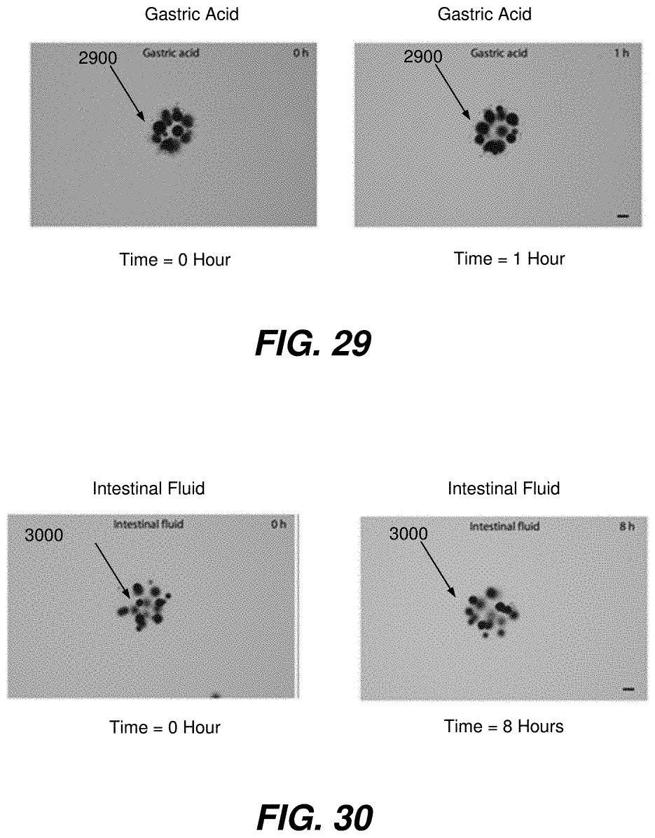

By implementing an enteric coating and gelatin encapsulation, an image-guided microrobotic device can have long-term stability in both gastric acid and intestinal fluid. FIG. 28 includes two microscopic images of image-guided microrobotic devices in gastric acid and intestinal fluid to show their stability, according to one implementation. FIG. 29 depicts two time-lapsed images taken at time=0 hour and 1 hour of an image-guided microrobotic device with an enteric coating and gelatin microcapsule 2900 in gastric acid to show stability, according to one implementation. FIG. 30 depicts two time-lapsed images taken at time=0 hour and 8 hours of an image-guided microrobotic device with an enteric coating and gelatin microcapsule 3000 in intestinal fluid to show stability, according to one implementation.

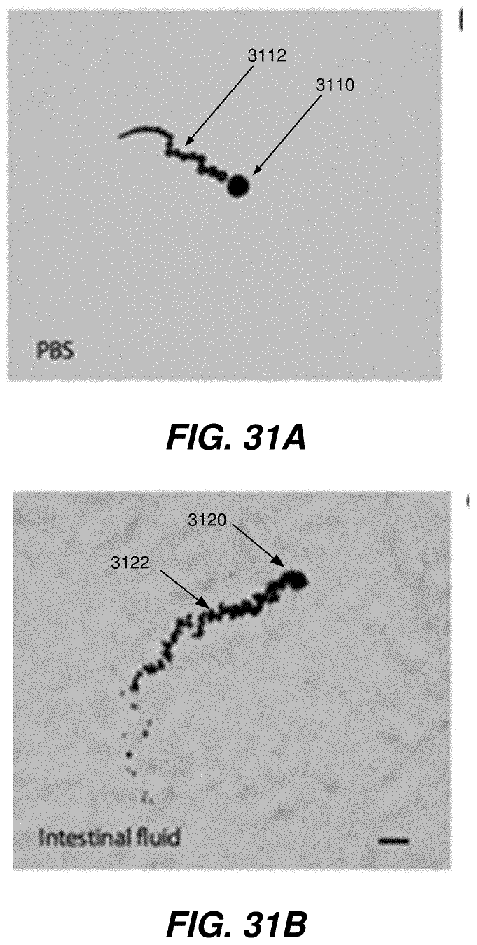

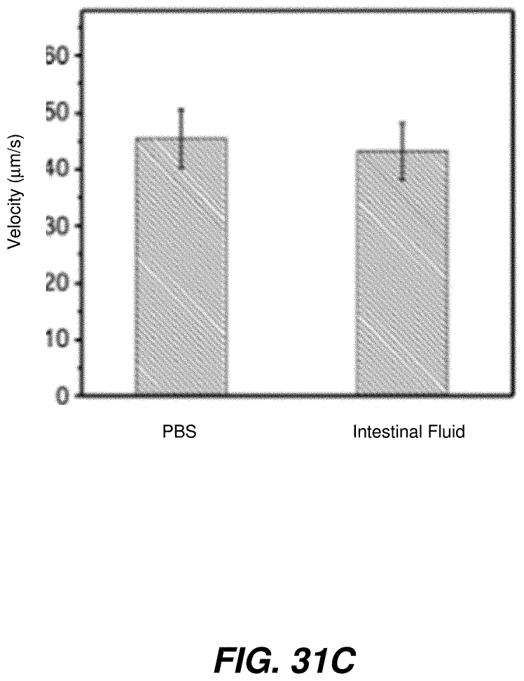

In certain aspects, micromotors exhibit gas bubble propulsion in various biofluids. FIG. 31A is a microscopic image showing the gas bubble propulsion of a micromotor 3110 in phosphate-buffered saline (PBS), according to an implementation. Gas bubbles 3112 are shown. FIG. 31B is a microscopic image showing the gas bubble propulsion of a micromotor 3120 in intestinal fluid, according to an implementation. Gas bubbles 3122 are shown. FIG. 31C is a bar graph showing the velocities of the micromotors 3110, 3120 in PBS and intestinal fluid, according to an implementation. Further quantitative analysis indicates that the velocities of the micromotors are 45 μm s⁻¹ and 43 μm s⁻¹ in PBS solution and the model intestinal fluid, respectively.

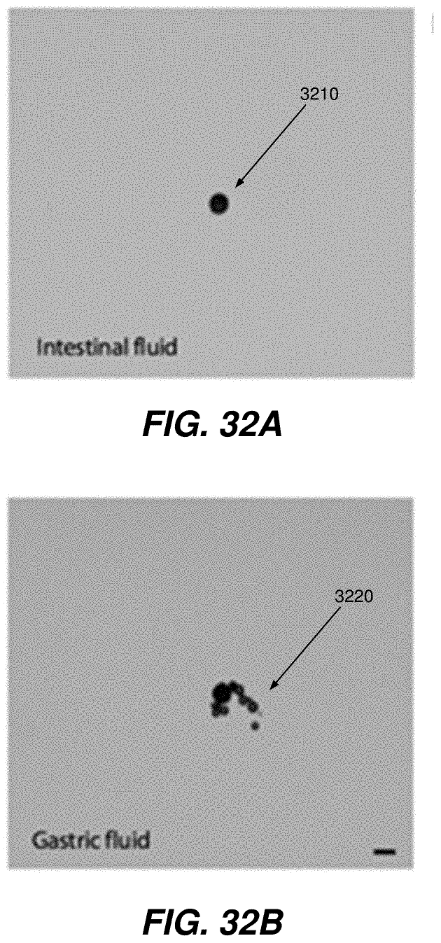

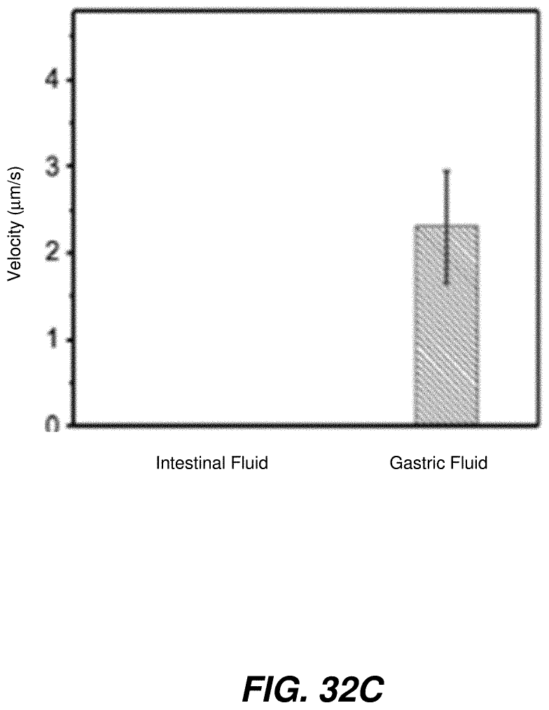

Bare Mg particles exhibit negligible propulsion in neutral media (e.g., intestinal fluid) and disordered propulsion in acidic conditions. FIG. 32A is a microscopic image showing the behavior of an Mg microparticle in intestinal fluid, according to an implementation. FIG. 32B is a microscopic image showing the behavior of an Mg microparticle in gastric acid, according to an implementation. FIG. 32C is a bar graph of the velocities of the micromotors 3110, 3120 in intestinal fluid and gastric fluid, according to an implementation. To simulate the gastric and intestinal environments, 0.01 M HCl (pH=2) was prepared as the model gastric fluid, and 50 mM potassium phosphate buffer (pH=6.5) was prepared as the model intestinal fluid. To characterize the movement of the micromotors, ˜10 μL of model fluid with 1% Triton X-100 were placed on a glass slide. Then, a ˜2 μL aqueous micromotor suspension in water was added into the model solution on the glass slide. The movement of micromotors was captured using a high-speed camera (Axiocam 720 mono) at ˜25 frames s⁻¹ and ImageJ with the plugin Manual Tracking was employed to track the micromotors.

Although CW-NIR irradiation is used in many examples herein to trigger collapse of the microcapsule(s), it would be understood that other triggering mechanisms in biomedicine, such as magnetic or ultrasonic fields, can also be employed to activate propulsion of the micromotors as discussed in Tay, Z. W., et al. “Magnetic particle imaging-guided heating in vivo using gradient fields for arbitrary localization of magnetic hyperthermia therapy,” ACS Nano 12, 3699-3713 (2018), which is hereby incorporated by reference in its entirety.

In Vivo Evaluation

The movement of a swarm of image-guided microrobotic devices according to one implementation was monitored in vivo using PACT (e.g., the PACT system 200 in FIG. 2). The image-guided microrobotic devices were dispersed in pure water and then orally administered into five (5) mice. The mice were subsequently anesthetized, and the lower abdominal cavity was aligned with the imaging plane of the ultrasonic transducer array for longitudinal imaging in a similar configuration as shown in FIG. 2. The PACT system captured time-lapse PACT images at a frame rate of 2 Hz for about 8 hours.

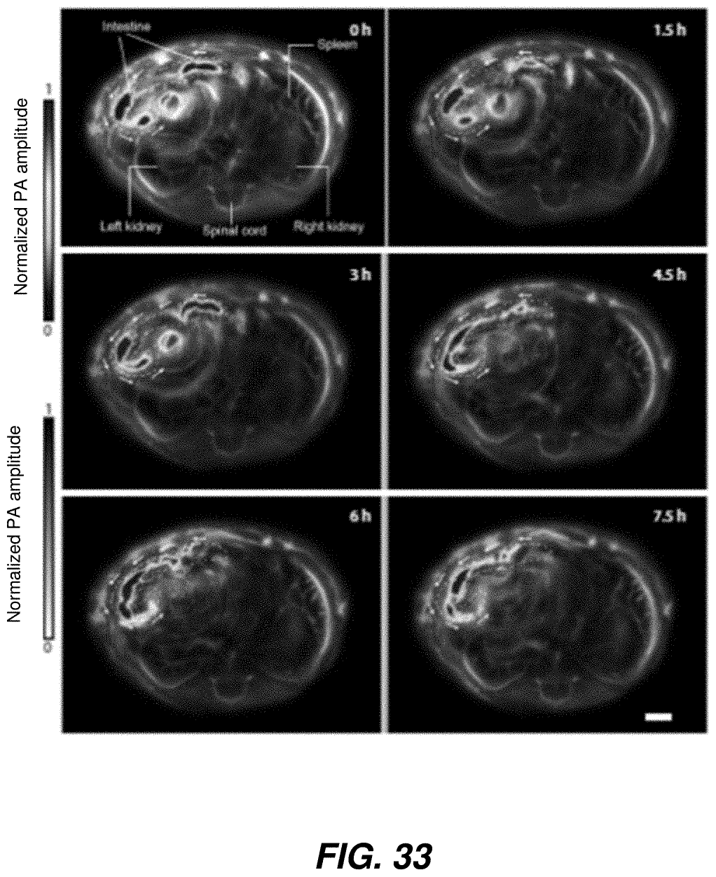

FIG. 33 includes gray-scaled versions of six (6) time-lapse PACT images of the image-guided microrobotic devices taken at time=0 hour, 1.5, hours, 3 hours, 4.5 hours, 6 hours, and 7.5 hours, according to an implementation. The blood vessels and background tissues are shown in gray and image-guided microrobotic devices in intestines are highlighted in (grayscaled) color. During the imaging period of the first 6 hours, the image-guided microrobotic devices migrated for about 1.2 cm, roughly 15% of the length of the entire small intestine. After 5 hours, the photoacoustic signals of some image-guided microrobotic devices faded away as they moved downstream in intestines that were outside of the imaging plane. The image-guided microrobotic devices were highlighted using temporal frequency filtering. The frames of interest were firstly smoothed by a Gaussian filter (6=3 pixels). Then Fourier transformation with respect to time was applied to all frames. An empirical band-pass filter was used to eliminate signals from either the static background or the respiration motion affected pixels, and thus the slowly moving pixels containing image-guided microrobotic devices were highlighted.

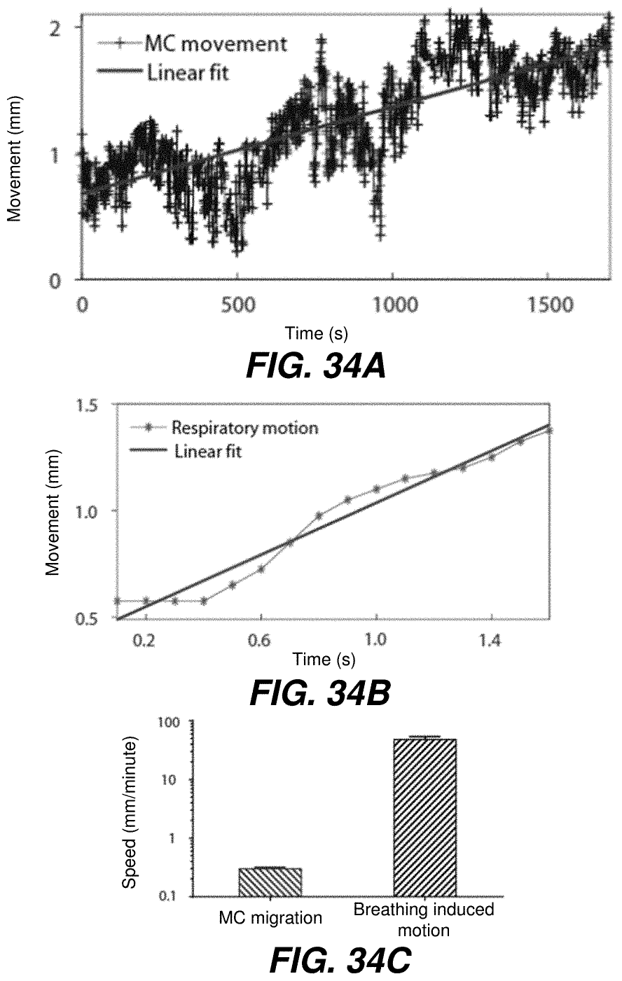

The moving speed of the swarm image-guided microrobotic devices in the intestines and the movements induced by respiratory motion were quantified. To quantify the speed of migration of the image-guided microrobotic devices, the acquired frames were first averaged to project the trajectories of the image-guided microrobotic devices. The migration paths of image-guided microrobotic devices were manually identified from the averaged image. Time traces at points along the migration paths were then extracted, forming images in which one dimension was the distance along the migration paths (x) and the other dimension was the elapsed time (t). Median filter (3×3 pixels) was then used to smooth the x-t images. Applying a threshold (⅓ of the maximum) segmented out the pixels containing image-guided microrobotic devices. The center positions of image-guided microrobotic devices along the path were estimated by calculating the geometric centers of the segmented pixels for given times. The center positions at the elapsed time points were fitted linearly to compute the migration speeds. FIG. 34A is a graph with a plot of the movement caused by the migration of the image-guided microrobotic devices in the intestine over time and a linear fit of the data, according to an implementation. FIG. 34B is a graph with a plot of the image-guided microrobotic device movement over time by the respiration motion of the mouse and a linear fit of the data, according to an implementation. FIG. 34C is a bar graph of a comparison of the speeds of the image-guided microrobotic devices migration and the respiration-induced movement. As shown in FIGS. 34A-C, the abrupt motion caused by respiration is much faster than actual migration of the image-guided microrobotic devices. Despite the respiration-induced movement, PACT can distinguish the signals from the slowly migrating image-guided microrobotic devices in the intestines, showing that PACT can precisely monitor and track the locations of the image-guided microrobotic devices in deep tissues in vivo.

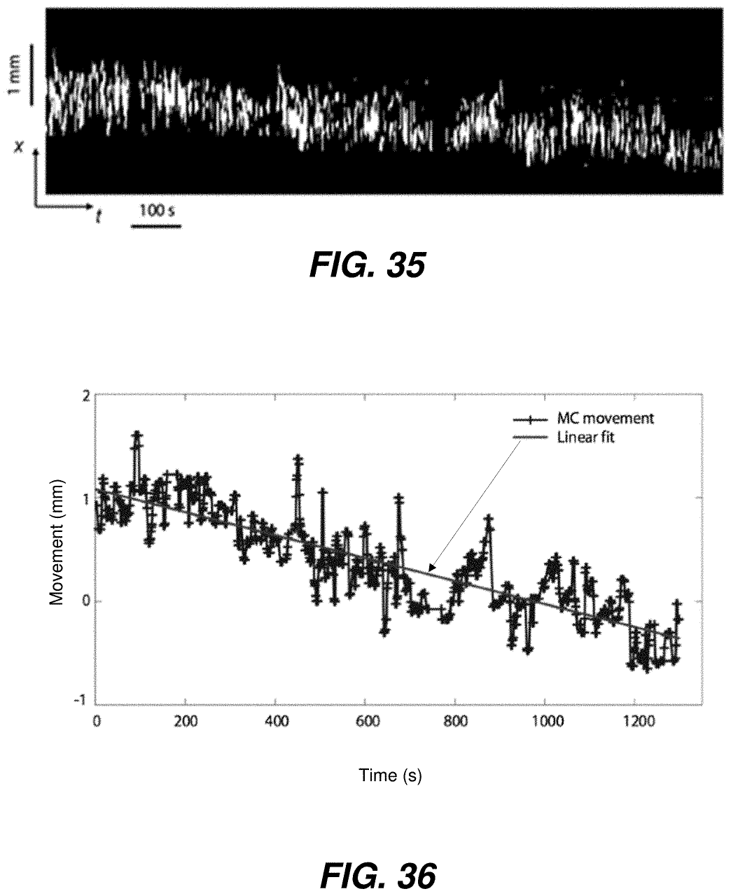

FIGS. 35 and 36 show quantification of image-guided microrobotic devices of certain aspects. FIG. 35 is a thresholded x-t image showing the segmented image-guided microrobotic devices at elapsed time, t, according to an example.

FIG. 36 is a graph of a plot of movement displacement caused by migration of the image-guided microrobotic devices in intestines, according to an example.

Retention Evaluation

The propulsion of cargo-loaded micromotors described herein may provide a mechanical driving force that can enhance their retention and delivery of cargo at or near targeted areas. In one aspect, the amount of NIR activation power needed to disintegrate the microcapsules may be adjusted by controlling the synthesis process and composition of the microcapsules. The amount of NIR activation power is depending on the mechanical properties of the microcapsule while the mechanical property could be controlled by the parameters during synthesis of the microcapsules.

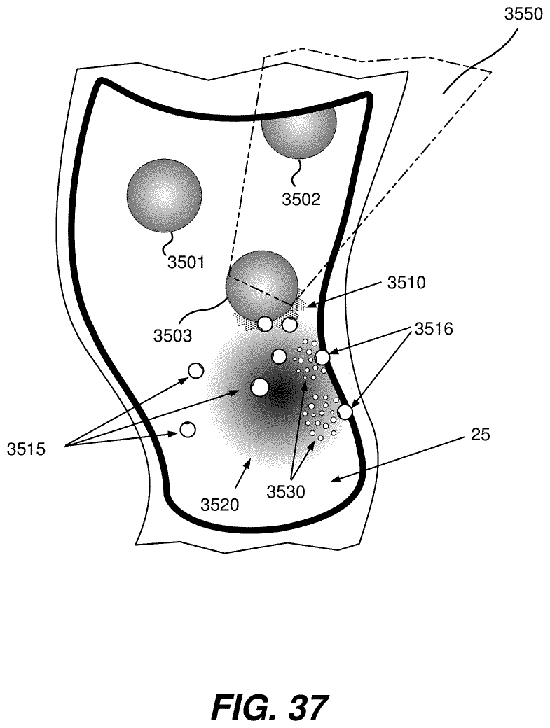

Of particular biomedical significance is the retention of cargo-loaded micromotors in a targeted region of the intestines. The biofluid-driven propulsion of active micromotors described herein may prolong retention in intestine walls. When the image-guided microrobotic devices approach the targeted areas of the intestines, the collapse of the microcapsules can be triggered and the propulsion of the micromotors activated on demand. FIG. 37 is schematic drawing of an implementation of using an image-guided microrobotic method for targeted delivery of micromotors in intestines, according to an implementation. As shown, image-guided microrobotic devices 3501, 3502, 3503 migrate down the intestine 25 as time-lapsed PACT images are taken. Using the images, the image-guided microrobotic system determines when the image-guided microrobotic device 3503 is approaching or at the targeted region 3520 and triggers the light source that directs the CW-NIR irradiation 3550 focused on the image-guided microrobotic device and/or the targeted region 3520. The CW-NIR irradiation 3550 induces the disintegration 3510 of the microcapsule of the image-guided microrobotic device 3503, which releases the micromotors 3515. When the reactive particles in the micromotors 3515 come into contact with the intestinal fluid, gas bubbles are generated and/or cargo 3530 released. The propulsion creates a mechanical driving force that causes two micromotors 3516 to enter or adhere to the intestinal wall at the targeted region 3520 of the intestine 25 to retain the micromotors 3516 at the targeted region 3520. This targeted retention of the micromotors 3516 may enable prolonged release (e.g. drug release) and retention of cargo 3530 at or near the targeted region 25.

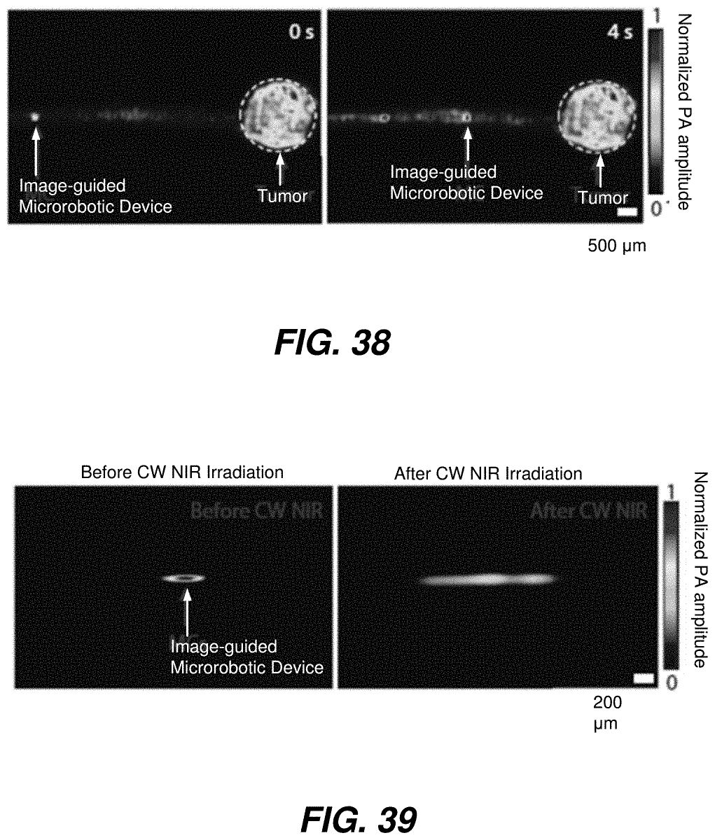

To investigate the use of the image-guided microrobotic methods for targeted delivery, melanoma cells were grown in mouse intestines and the intestines were coated with tissues as the model ex vivo colon tumor. Due to the high optical absorption of melanoma cells in the NIR wavelength region, colon tumors can be clearly resolved by PACT. After injection into the intestines, the image-guided microrobotic devices migrated toward the targeted colon tumor. A syringe pump was also connected to drive the image-guided microrobotic devices. FIG. 38 depicts two time-lapsed PACT images at time=0 and 4 seconds of the migration of an image-guided microrobotic device toward the model colon tumor, according to an implementation.

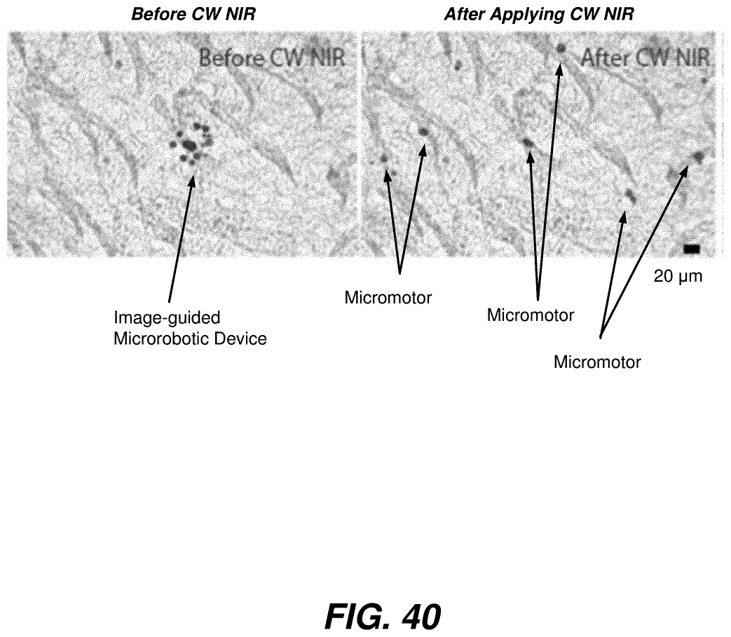

Once the image-guided microrobotic devices approached the targeted region, they were irradiated with CW NIR light to trigger a responsive release of the micromotors. The photoacoustic signals from the image-guided microrobotic devices in the intestines were prolonged upon the CW NIR irradiation, suggesting the release of the micromotors. FIG. 39 depicts two images 1) first image with an image-guided microrobotic device before activation by WC NIR irradiation and 2) second image after activation by CW NIR irradiation, according to an implementation. FIG. 40 depicts two overlaid microscope images one before activation by WC NIR irradiation and one after activation by CW NIR irradiation, according to an implementation. The overlaid microscopic images in FIG. 40 show the NIR-triggered release of the micromotors from an MC in the intestines. The DOX-loaded micromotors, clearly observed with red fluorescence, rapidly diffused into the surrounding area after the CW NIR irradiation.

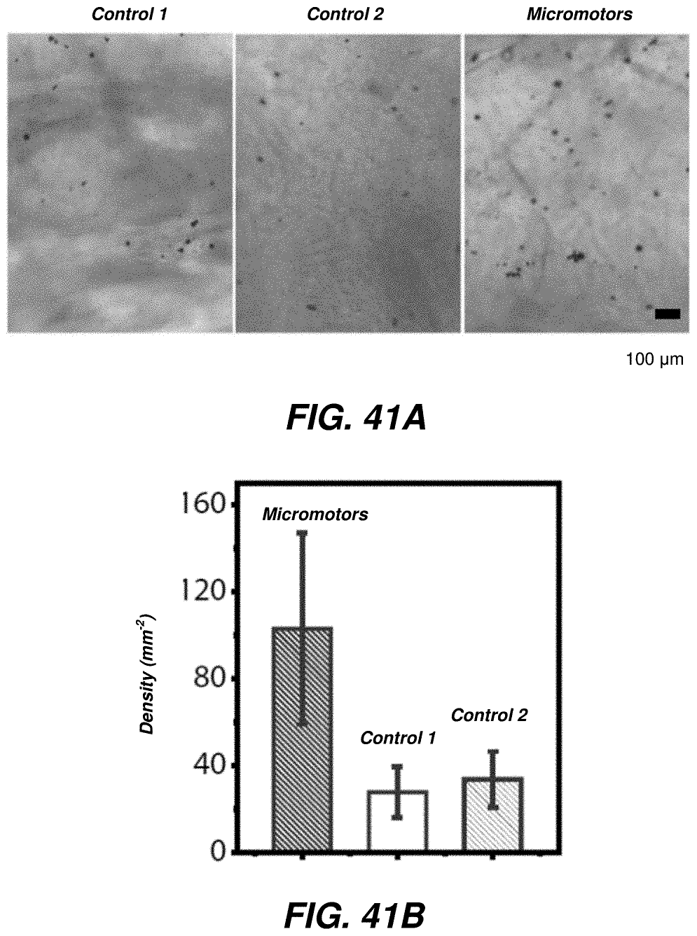

To evaluate retention of the micromotors in vivo, the micromotors encapsulated in enteric polymer-coated microcapsules and paraffin-coated passive Mg and Mg/Au particles (as Control 1 and Control 2 respectively) were orally administrated to three mouse groups. As the controls, paraffin-coated passive particles (Mg particles and Mg/Au particles as Control 1 and Control 2, respectively) were prepared by incubating 0.05 g particles with 1 g paraffin wax at 75° C. overnight and then sequentially washed with chloroform, acetone, and pure water as discussed in Hong, L., Jiang, S., Granick, S., “Simple method to produce Janus colloidal particles in large quantity,” Langmuir 22, 9495-9499 (2006), which is hereby incorporated by reference in its entirety. The intestines from the mice treated with micromotors retained a much higher number of micromotors than that with passive particles. FIG. 41A depicts three microscopic images showing the in vivo retention of the control microparticles and the micromotors in intestines, according to an implementation. Control 1 represents the paraffin-coated passive Mg microparticles. Control 2 represents the paraffin-coated passive Mg/Au microparticles. The quantitative analysis displays a 3- to 4-fold increase in the density of the micromotors in the treated intestine segments. FIG. 41B is a bar graph of the density of particle micromotor retention in intestines of the micromotors, according to an implementation. Compared with control samples, a higher amount of micromotors was found in intestine in FIG. 41B.

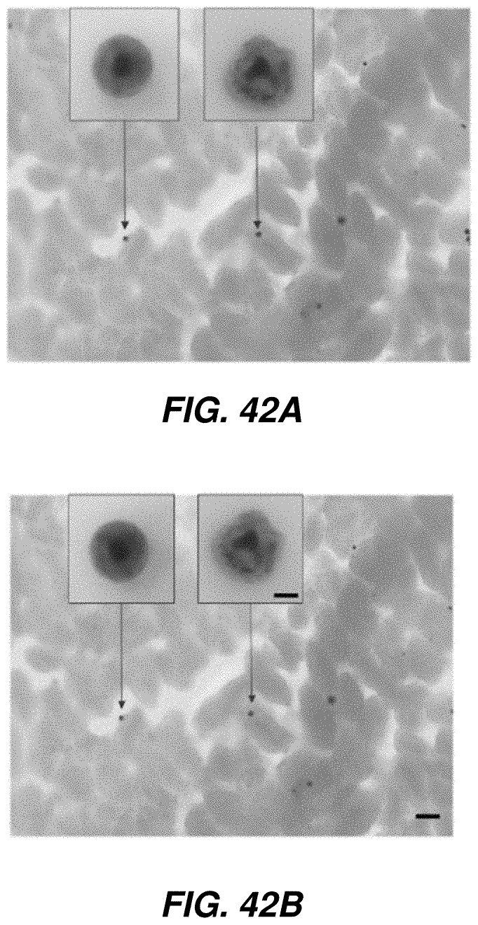

The images show hollow structures of the micromotors in the intestine before and after acid treatment. is a microscopic image of the micromotors attached to the intestines before the addition of 0.1 M gastric acid, according to an implementation. FIG. 42A is a microscopic image showing micromotors attached on the intestines before addition of 0.1 M gastric acid, according to an implementation. FIG. 42B is a microscopic image showing micromotors attached on the intestines after addition of 0.1 M gastric acid, according to an implementation. Insets show enlarged images of the micromotors. FIG. 42B illustrates that the magnesium part of the micromotors can degrade after 12 hours in vivo.

Besides active propulsion, the enhanced retention in vivo may also be attributed to the elevated pH and Mg²⁺ concentration in the surrounding environment caused by Mg-water reactions in certain implementations. The enhanced retention of micromotors in vivo may be attributed to the interaction between micromotors and intestinal mucus. The chemical reaction of magnesium and water generated Mg²⁺ and elevated pH in local environment, which may trigger the phase transition of mucus according to Tay, Z. W., et al. “Magnetic particle imaging-guided heating in vivo using gradient fields for arbitrary localization of magnetic hyperthermia therapy,” ACS Nano 12, 3699-3713 (2018) and Bansil, R., Turner, B. S., “The biology of mucus: Composition, synthesis and organization,” Adv. Drug Deliv. Rev. 124, 3-15 (2018), which are hereby incorporated by reference in their entireties. High pH (˜8.2-12.0) could trigger a phase transition of the mucus and facilitate tissue penetration of the micro/nanoparticles as discussed in Tay, Z. W., et al. “Magnetic particle imaging-guided heating in vivo using gradient fields for arbitrary localization of magnetic hyperthermia therapy,” ACS Nano 12, 3699-3713 (2018), Bansil, R., Turner, B. S., “The biology of mucus: Composition, synthesis and organization,” Adv. Drug Deliv. Rev. 124, 3-15 (2018), Celli, J. P., et al., “Helicobacter pylori moves through mucus by reducing mucin viscoelasticity,” Proc. Natl. Acad. Sci. U.S.A 106, 14321-14326 (2009), Lai, S. K., Wang, Y.-Y., and Hanes, J., “Mucus-penetrating nanoparticles for drug and gene delivery to mucosal tissues,” Adv. Drug Deliv. Rev. 61, 158-171 (2009), which are hereby incorporated by reference in their entireties.

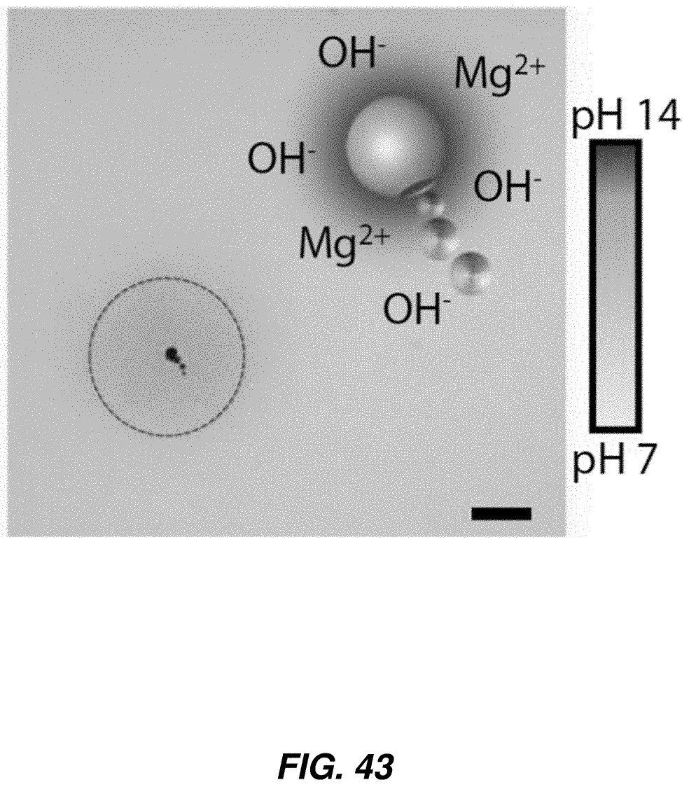

To investigate the influence of the micromotors on the pH of the surrounding environment, the micromotors were dispersed in water with phenolphthalein as a pH indicator. FIG. 43 is a microscopic image (lower left) and a schematic drawing (upper right) illustrating the change of pH of the surrounding environment upon the micromotors being released into PBS, according to an implementation. The microscopic image shows a dark portion in the vicinity of a micromotor, indicating an increased pH of the surrounding medium. In addition, an increased concentration of divalent cation Mg²⁺ can cause collapse of the mucus gel as discussed in Leal, J., Smyth, H. D. C., Ghosh, D., “Physicochemical properties of mucus and their impact on transmucosal drug delivery,” Int. J. Pharm. 532, 555-572 (2017), which is hereby incorporated by reference in its entirety.

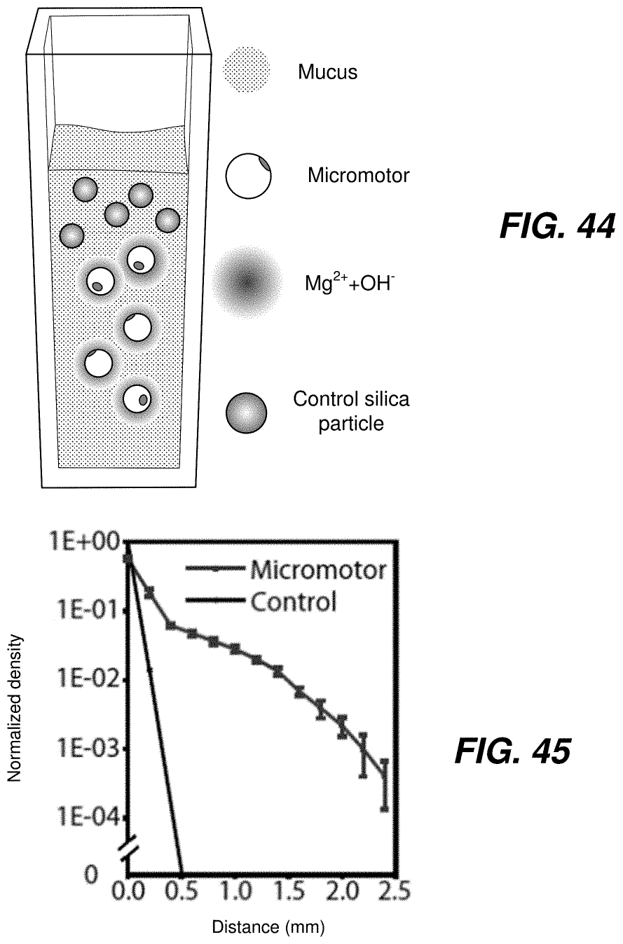

The enhanced diffusion of the micromotors in mucus was further investigated using a technique discussed in Kirch, J., et al., “Optical tweezers reveal relationship between microstructure and nanoparticle penetration of pulmonary mucus,” Proc. Natl. Acad. Sci. U.S.A 109, 18355-18360 (2012), which is hereby incorporated by reference in its entirety. FIG. 44 is a schematic drawing of the control silica particles and the ingestible micromotors in mucus after 1 hour, according to an implementation. The drawing shows the reaction Mg²⁺+OH⁻ at the micromotors. A cuvette was filled with 3.5 mL porcine mucus, and then a 100 μL micromotors suspension (˜10⁶ mL⁻¹ in water) was pipetted into the mucus. Silica microparticles of the same size were utilized as control. Optical images were captured every 2 minutes. During the observation, the cuvettes were treated with sonication for 5 seconds with an ultrasound bath cleaner to remove bubbles. ImageJ was employed to count particles in the mucus. The numbers were normalized by the number of particles injected at the start, and the ratios were calculated at distances away from the initial point. FIG. 45 are the diffusion profiles of the control silica particles and the ingestible micromotors, according to an implementation.

Encapsulation and Release of Drug from Micromotors

In one aspect, the encapsulation efficiency (EE) and release profile of DOX for image-guided microrobotic devices and micromotors can be increased using techniques described in Cui, Y., et al. “Transferring-conjugated magnetic silica PLGA nanoparticles loaded with doxorubicin and paclitaxel for brain glioma treatment,” Biomaterials 34, 8511-8520 (2013) and Gaihre, B., Khil, M. S., Lee, D. R., Kim, H. Y., “Gelatin-coated magnetic iron oxide nanoparticles as carrier system: Drug loading and in vitro drug release study,” Int. J. Pharm. 365, 180-189 (2009), which are hereby incorporated by reference in their entireties. To encapsulate DOX into the micromotors, 1.0 mL alginate solution (2%, w/v) with different concentrations of DOX were dropped onto the glass slides containing Au layer-coated Mg microparticles, and then a 1.0 mL CaCl₂) solution was dropped onto the glass slide to cross-link alginate, followed by coating of a parylene layer and water rinse for 3 times. Micromotors without cross-linking were also prepared. The amount of DOX was measured through a UV-visible spectrophotometer at 485 nm. The EE of DOX on the micromotors can be determined using the following equation:

$$EE of DOX ( \% ) = \frac{\begin{matrix}Initial amount of DOX used - \\ amount of DOX in supernatant\end{matrix}}{Initial amount of DOX used} \times 100 \% \qquad ( Eqn . 1 )$$

For the drug release study, ˜10 mg DOX-loaded micromotors were suspended in 5 mL PBS with magnetic stirring at 37° C. and 8000 rpm. At different time intervals, the supernatant was removed and replaced with fresh PBS. The concentration of DOX was determined by measuring its absorbance using a spectrophotometer at a wavelength of 485 nm.

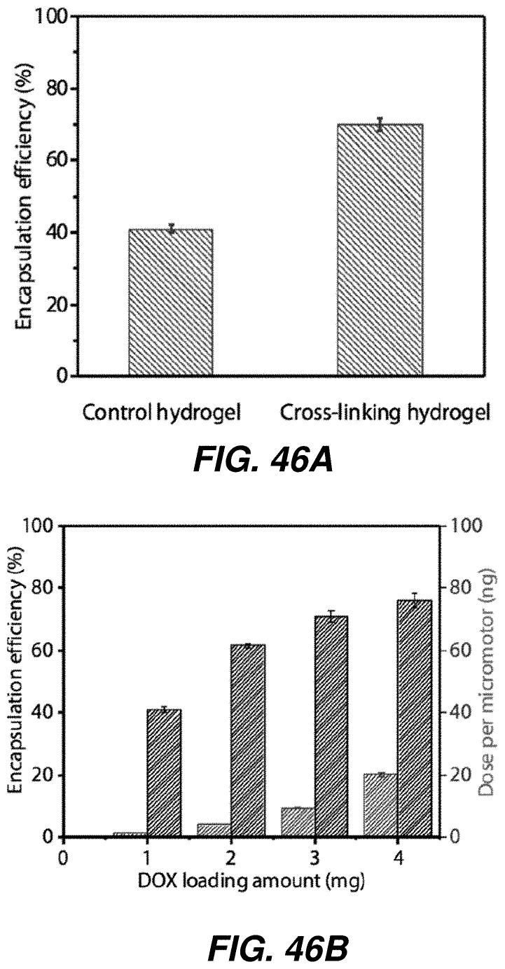

Compared with the negligible diffusion of the control silica particles in the mucus, diffusion of the micromotors in the mucus shows a significantly enhanced profile within 40 minutes. To investigate the cargo release kinetics of the micromotors, a fluorescent anticancer drug, DOX, was encapsulated into the alginate layer of the micromotors. The release of DOX from the micromotors was characterized utilizing an ultra-violet/visible spectrophotometer. The cross-linking treatment of the hydrogel significantly improves the efficiency of DOX loading. FIGS. 46A and 46B show the effects of cross-linking. FIG. 46A is a bar graph of encapsulation efficiency for control hydrogel and cross-linking hydrogel, according to an implementation. FIG. 46B is a bar graph of encapsulation efficiency vs. DOX loading amount per micromotor, according to an implementation. By increasing the DOX loading amount from 0.5 to 4 mg, the dose of DOX per micromotor can be controlled from ˜1 to 20 ng while the encapsulation efficiency can be improved up to 75.9% as shown in FIG. 46B.

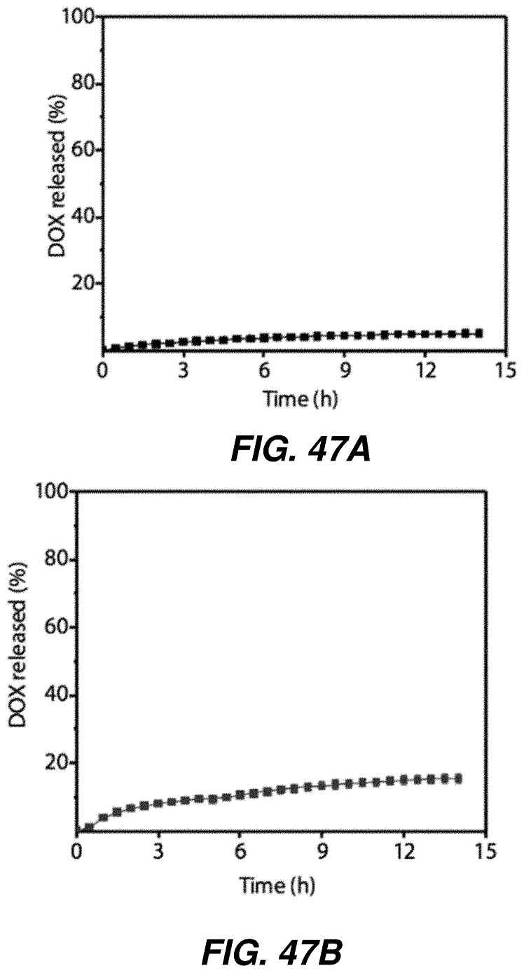

FIG. 47A is graph with a plot of DOX released percentage from image-guided microrobotic devices as a function of time, according to an implementation. FIG. 47B is graph with a plot of DOX released percentage from micromotors as a function of time, according to an implementation. As shown in FIGS. 47A and 47B, a higher release rate was observed in the DOX-loaded micromotors in comparison to the DOX-loaded image-guided microrobotic devices. These results may show promise of using the micromotors for in vivo targeted therapy of GI diseases such as colon cancer.

Biocompatability and Biodegradablity of Image-Guided Microrobotic Devices

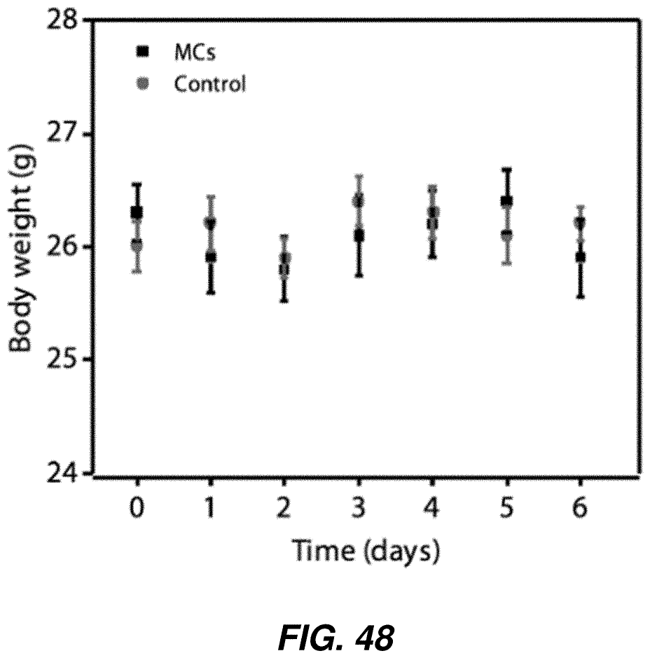

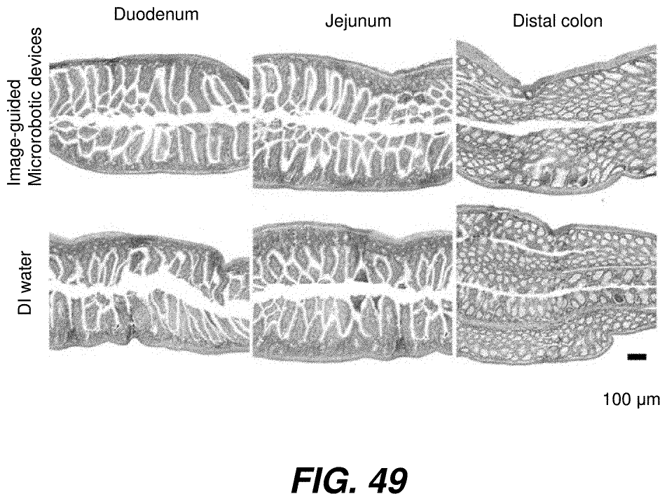

The biocompatibility and biodegradability of the image-guided microrobotic devices are important for biomedical applications. The materials of the image-guided microrobotic devices, such as Mg, Au, gelatin, alginate, and enteric polymer are known to be biocompatible. To evaluate the toxicity profile of the image-guided microrobotic devices in vivo, healthy mice were orally administered with image-guided microrobotic devices or DI water once a day for two consecutive days. Throughout the treatment, no signs of distress, such as squinting of eyes, hunched posture, or lethargy, were observed in either group. Initially, the toxicity profile of the image-guided microrobotic devices in mice was evaluated through changes in body weight. During the experimental period, the body weights of the mice administered with image-guided microrobotic devices have no significant difference from those of the control group. FIG. 48 is a graph of the body weight changes in mice after oral administration of the image-guided microrobotic devices and the control (DI water) over time, according to an implementation. A histology analysis was performed to evaluate further the toxicity of the image-guided microrobotic devices in vivo. No lymphocytic infiltration into the mucosa or submucosa was observed, indicating no signs of inflammation. FIG. 49 is a histology analysis for the duodenum, jejunum, and distal colon of the mice treated with the image-guided microrobotic devices or DI water as the control for 12 hours, according to an implementation.

The components of micromotors described herein are widely used as therapeutic agents and in implantable devices have been studied to be safe for in vivo applications as discussed in Smith, B. R., Eastman, C. M., Njardarson, J. T., “Beyond C, H, 0, and Ni analysis of the elemental composition of U.S. FDA approved drug architectures,” J. Med. Chem. 57, 9764-9773 (2014) and Baheiraei, N., Azami, M., Hosseinkhani, H., “Investigation of magnesium incorporation within gelatin/calcium phosphate nanocomposite scaffold for bone tissue engineering,” Int. J. Appl. Ceram. Technol. 12, 245-253 (2015) and Sezer, N., Evis, Z., Kayhan, S. M., Tahmasebifar, A. Koç, M., “Review of magnesium-based biomaterials and their applications,” J. Magnesium Alloys 6, 23-43 (2018), which are hereby incorporated by reference in their entireties. The micromotors have been shown to be eventually cleared by the digestive system via excrement, without any adverse effects.

To estimate the toxicity of the image-guided microrobotic devices in vivo, 5-6-week old nude mice were administered with 0.1 mL micromotor suspension via oral gavage. Healthy mice treated with DI water were used as a negative control. The body weight of mice was measured daily during the experiment. In order to prepare the intestine sample for histology investigation, the intestines were treated with 10% (v/v) buffered formalin for 15 hours. The intestines were cut to smaller sections as duodenum, jejunum, and distal colon. The longitudinal tissue sections were washed in tissue cassettes and embedded in paraffin. The tissue sections were sliced into 8-μm thick sections using a freezing microtome (Leica, CM1950) and stained with H&E assay. The samples were imaged with an optical microscope (Zeiss, AXIO).

Penetration Depth with Different Imaging Techniques

In human clinical applications, tissue penetration may be up to tens of centimeters. PACT can provide up to 7-cm tissue penetration, which is limited by photon dissipation. In some implementations, the image-guided microrobotic methods/systems employ imaging techniques that use excitation sources such as, e.g., microwave, acoustic detection, and thermoacoustic tomography (TAT) that are capable of tissue penetration for human clinical applications as discussed in Xu, Y., Wang, L. V., “Rhesus monkey brain imaging through intact skull with thermoacoustic tomography,” IEEE Trans. Ultrason. Ferroelectr. Freq. Control 53, 542-548 (2006) and Kruger, R. A., et al. “Thermoacoustic CT: imaging principles,” Proc. SPIE 3916, 150-160 (2000), which are hereby incorporated by reference in their entireties. In implementations that use a gold layer as the imaging contrast layer in the micromotor, the gold layer may provide an excellent microwave absorption contrast for TAT imaging owing to the high electrical conductivity, and thus greatly enhances the deep tissue imaging capability of the micromotors. Focused ultrasound heating may also increase the depths of thermally-triggered microrobot release to the whole-body level of humans.

Applications Other than Intestines

Passive diffusion-based targeting strategies have been explored to improve delivery efficiency, but they suffer from low precision, size restraint and specific surface chemistry as discussed in Rosenblum, D., Joshi, N., Tao, W., Karp, J. M., Peer, D., “Progress and challenges towards targeted delivery of cancer therapeutics,” Nat. Commun. 9, 1410 (2018), which is hereby incorporated by reference in its entirety.

Certain implementations of image-guided microrobotic techniques described herein enable micromotors to reach a targeted region in intestines with high precision. These techniques can be tailored to reactive particles of various sizes and can be applied to any biological media such as, for example, gastrointestinal tract, blood, urea, and interstitial fluid. In one aspect, reactive particles are in a range of 3 μm to 1 mm. In another aspect, reactive particles are in a range of 20 μm to 60 μm. The image-guided microrobotic techniques can implement micromotors with material that can carry various cargos such as, e.g., therapeutic agents and diagnostic sensors, with real-time feedback during delivery to the target region and activation.

Certain implementations of image-guided microrobotic techniques described herein pertain to an ingestible image-guided microrobotic device with high optical absorption for imaging-assisted control in, e.g., intestines. The encapsulated micromotors survive the erosion of the stomach fluid and permit propulsion in intestines. In one aspect, PACT non-invasively monitors the migration of the micromotors and visualizes their arrival at targeted areas in vivo. As the micromotors arrive at or near the targeted region, CW NIR irradiation may be used to induce a phase transition of the microcapsules and trigger the propulsion of the micromotors. The mechanical propulsion provides a driving force for the micromotors to bind to the intestine walls, resulting in an extended retention.

Modifications, additions, or omissions may be made to any of the above-described embodiments without departing from the scope of the disclosure. Any of the embodiments described above may include more, fewer, or other features without departing from the scope of the disclosure. Additionally, the steps of described features may be performed in any suitable order without departing from the scope of the disclosure. Also, one or more features from any embodiment may be combined with one or more features of any other embodiment without departing from the scope of the disclosure. The components of any embodiment may be integrated or separated according to particular needs without departing from the scope of the disclosure.

It should be understood that certain aspects described above can be implemented in the form of logic using computer software in a modular or integrated manner. Based on the disclosure and teachings provided herein, a person of ordinary skill in the art will know and appreciate other ways and/or methods to implement the present invention using hardware and a combination of hardware and software.

Any of the software components or functions described in this application, may be implemented as software code using any suitable computer language and/or computational software such as, for example, Java, C, C#, C++ or Python, LabVIEW, Mathematica, or other suitable language/computational software, including low level code, including code written for field programmable gate arrays, for example in VHDL. The code may include software libraries for functions like data acquisition and control, motion control, image acquisition and display, etc. Some or all of the code may also run on a personal computer, single board computer, embedded controller, microcontroller, digital signal processor, field programmable gate array and/or any combination thereof or any similar computation device and/or logic device(s). The software code may be stored as a series of instructions, or commands on a CRM such as a random access memory (RAM), a read only memory (ROM), a magnetic medium such as a hard-drive or a floppy disk, or an optical medium such as a CD-ROM, or solid stage storage such as a solid state hard drive or removable flash memory device or any suitable storage device. Any such CRM may reside on or within a single computational apparatus, and may be present on or within different computational apparatuses within a system or network. Although the foregoing disclosed embodiments have been described in some detail to facilitate understanding, the described embodiments are to be considered illustrative and not limiting. It will be apparent to one of ordinary skill in the art that certain changes and modifications can be practiced within the scope of the appended claims.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

What is claimed is:

1. A microrobotic device, comprising:
one or more micromotors, each micromotor comprising:
a reactive particle;
a partial coating disposed on the reactive particle, the partial coating comprising:
an imaging contrast layer;
a cargo layer; and
an encapsulation layer; and
a microcapsule encapsulating the one or more micromotors.

2. The microrobotic device of claim 1, wherein the partial coating includes one or more areas open to the reactive particle.

3. The microrobotic device of claim 1, wherein at least one of the micromotors is configured to generate propulsion when in contact with a fluid.

4. The microrobotic device of claim 1, wherein the imaging contrast layer or the cargo layer is disposed on the reactive particle.

5. The microrobotic device of claim 1, wherein the imaging contrast layer comprises one or more metals.

6. The microrobotic device of claim 1, wherein the imaging contrast layer comprises gold.

7. The microrobotic device of claim 6, wherein the imaging contrast layer has a thickness in a range of 1 μm to 20 μm.

8. The microrobotic device of claim 1, wherein the partial coating comprises a magnetically-charged material.

9. The microrobotic device of claim 1, wherein the cargo layer comprises a gelatin hydrogel material.

10. The microrobotic device of claim 1, wherein the cargo layer comprises a drug and/or an imaging contrast agent.

11. The microrobotic device of claim 1, wherein the encapsulation layer comprises parylene.

12. A method of fabricating a microrobotic device, the method comprising:
fabricating one or more micromotors, each micromotor fabricated by depositing a partial coating on a reactive particle, the partial coating comprising an imaging contrast material and cargo, the partial coating having one or more areas open to the reactive particle; and
encapsulating the one or more micromotors in a microcapsule.

13. The method of claim 12, wherein the method comprises:
depositing an imaging contrast layer;
depositing a cargo layer; and
depositing an encapsulation layer.

14. The method of claim 12, wherein the method comprises generating at least one of the open areas by surface contact of the reactive particle with a glass surface during deposition of the partial coating.

15. The method of claim 12, wherein the one or more micromotors are encapsulated by an emulsion operation.

16. An image-guided microrobotic method, comprising:
using one or more images to determine that a microrobotic device is at or near a target region, wherein the microrobotic device comprises one or more micromotors encapsulated in a microcapsule, at least one of the micromotors comprising a partial coating disposed over a reactive particle, the partial coating comprising an imaging contrast material and cargo;
inducing disintegration of at least a portion of the microcapsule.

17. The image-guided microrobotic method of claim 16, wherein disintegration is induced by applying one of near-infrared irradiation, high-intensity focused ultrasound, or magnetic field.

18. The image-guided microrobotic method of claim 16, wherein the partial coating includes one or more areas open to the reactive material.

19. The image-guided microrobotic method of claim 16, further comprising received the one or more images were constructed using one of photoacoustic computed tomography, magnetic resonance imaging, and ultrasound.

20. The image-guided microrobotic method of claim 16, further comprising using photoacoustic computed tomography to generate the one or more images by:
causing a pulsed light source to generate one or more light pulses configured to illuminate a specimen being imaged, the specimen having the target region;
controlling a scanning mechanism to move and/or scan the ultrasonic transducer array in a direction along an axis, wherein the ultrasonic transducer array includes a plurality of unfocused transducer elements, wherein the ultrasonic transducer array is moved/scanned in the direct along the axis while each of a plurality of unfocused transducer elements detects photoacoustic waves within a field-of-view in a range of 5 degrees to 30 degrees in the direction along the axis; and
reconstructing the one or more images using photoacoustic signals recorded while the scanning mechanism moves/scans the ultrasonic transducer array in the direction along the axis.
